(19) United States
(12) Patent Application Publication
(10) Pub. No.: US 2009/0192095 A1
(43) Pub. Date: Jul. 30, 2009

(54) AMINO ACID AND PEPTIDE PRODRUGS OF OPIOID ANALGESICS WITH REDUCED GI SIDE-EFFECTS

(75) Inventors: Richard Franklin, Hampshire (GB); Bernard T. Golding, Newcastle upon Tyne (GB); Robert G. Tyson, Durham (GB)

Correspondence Address: DARBY & DARBY P.C., P.O. BOX 770, Church Street Station, New York, NY, 10008-0770, US

(73) Assignee: SHIRE LLC, Florence, KY (US)

(21) Appl. No.: 12/356,034

(22) Filed: Jan. 19, 2009

Related U.S. Application Data
(60) Provisional application No. 61/022,044, filed on Jan. 18, 2008.
(60) Provisional application No. 61/022,159, filed on Jan. 18, 2008.

(51) Int. Cl. A61K 38/05; A61K 31/485; A61K 31/55; A61K 31/4355; C07D 223/04; C07D 489/08; C07D 491/08; A61P 1/00

(52) U.S. Cl. 514/19; 514/282; 514/212.01; 514/279; 540/611; 546/45; 546/39

(57) ABSTRACT

The present invention relates to methods for reducing gastrointestinal side effects in a subject, the gastrointestinal side effects being associated with the administration of an opioid analgesic. The methods comprise orally administering an opioid prodrug or pharmaceutically acceptable salt thereof to a subject, wherein the opioid prodrug is comprised of an opioid analgesic covalently bonded through a carbamate linkage to a peptide of 1-5 amino acids in length, and wherein upon oral administration, the prodrug or pharmaceutically acceptable salt minimizes at least one gastrointestinal side effect associated with oral administration of the opioid analgesic alone. Compositions for use with the method are also provided.

This application claims the benefit of U.S. Provisional Application Nos. 61/022,044 and 61/022,159, both filed Jan. 18, 2008. These prior applications are hereby incorporated by reference.

### FIELD OF THE INVENTION

The present invention relates to the utilization of prodrugs of opioid analgesics to reduce the opioid analgesic's adverse gastrointestinal (GI) side effects, including constipation and vomiting.

### BACKGROUND OF THE INVENTION

Appropriate treatment of pain continues to represent a major problem for both subjects and healthcare professionals. Optimal pharmacologic management of pain requires selection of the appropriate analgesic drug that achieves rapid efficacy with minimal side effects.

Analgesics for treating mild pain are readily available, both over the counter (OTC) and by prescription. These include aspirin, ibuprofen and acetaminophen (paracetamol). While these agents are well established for the treatment of mild pain, they are not without their side effects. For example, aspirin may cause local stomach irritation and paracetamol, in excessives doses, is associated with marked liver toxicity followed potentially by liver failure.

More effective analgesics such as the stronger non-steroidal anti inflammatory drugs, (e.g., ketoprofen, diclofenac and naproxen), while offering effective pain relief in moderate pain, may have more pronounced side effects such as gastric ulceration and possible hemorrhage.

Treatment of more severe pain with opioid analgesics such as oxyocodone, oxymorphone, hydromorphone and morphine offers good analgesia, but is beset by problems of gastrointesinal (GI) tract intolerance and adverse reactions. These adverse GI reactions include nausea, dyspepsia, vomiting, gastric ulceration, diarrhea and constipation, or, in some cases, a combination of these reactions.

Peptide prodrugs of various opioids have been synthesized previously and are described in, for example, International Patent Application Publication Nos. WO 05/032474, WO 07/126,832 and WO 02/034237, WO 03/020200, WO 03/072046, WO 07/030,577 and WO 2007/120648. However, it is unknown if these prodrugs can alleviate gastrointestinal side effects associated with unmodified opioid molecules.

Constipation represents the single most serious GI side effect associated with opioid use. It has been estimated that between 40-63% of patients receiving opioids for chronic pain suffer from so-called “opioid induced bowel dysfunction,” the most common symptom of which is constipation (Panchal et al. (2007). Int J Clin Pract 61, 1181-1187). However, such patients experience a wide range of symptoms including decreased gastric emptying (exacerbating any pre-existing hiatus hernia and propensity to oesophageal reflux and oesophagitis), abdominal cramping, bloating, spasm, delayed GI transit and the formation of hard stools. The latter can be particularly problematic in a post operative setting following major abdominal or gyneacological surgery. Straining at stool in the immediate post operative period can have serious consequences for the integrity of the surgical wound and may necessitate further restorative surgery.

Furthermore, the adverse effects of opioids on gut motility represent a major contributing factor to the development of post operative ileus (POI). POI can affect patient mobility and can include abdominal distention, pain, nausea, vomiting, inability to pass stools and inability to tolerate a solid diet. POI is one of the most common reasons for hospital re-admission and is a major economic burden on national healthcare systems. For example, in the United States, it has been reported that POI complication adds between $50,000,000 to $1,280,000,000 to annual healthcare costs (Linn and Steinbrook (2007). Tech. in Regional Anaes. and Pain Mangmt 11, 27-32). A recent analysis of some 1,050,558 patients undergoing open appendectomy found that the cost associated with POI effectively doubled mean hospital charges from $12,924 to $24,767 per hospitalization (Linn and Steinbrook (2007). Tech. in SRegional Anaes. and Pain Mangmt 11, 27-32).

For the older patient, frequently the target for stronger analgesic use, chronic constipation is a common side effect and one of particular concern. Constipation will inevitably exacerbate any pre-existing problem of hemorrhoids and may induce rectal bleeding and local itching, thereby adversely affecting the quality of life. Such uncomfortable side-effects can lead to compliance problems, ineffective medication and therefore, lack of pain relief. Unlike the nausea and vomiting associated with opioid treatment, which may in part be ameliorated with anti-emetic co-therapy, opioid induced bowel dysfunction is neither easily nor effectively treatable.

Opioid induced bowel dysfunction invariably involves treatment with stool softeners and laxatives, such as Movicol™. However, such laxatives are frequently ineffective, especially in those patients requiring increasingly higher doses of opioids. It has been found that a significant number of patients would rather endure their pain than suffer the incapacitating effects of chronic constipation, an enlightening measure of the severity and distress that this problem causes (Vanegas (1998). Cancer Nursing 21, 289-297).

Vomiting is also a side effect associated with opioid administration. The act of vomiting can increase intracranial pressure, increase the risk of further damage to patients with ocular injuries and abdominal wounds, and impact on vagal stimulation causing changes in blood pressure and pulse rate. The powerful muscular contractions associated with vomiting may cause further damage in specific instances of acute pain. Vomiting can also cause dehydration and regurgitation of stomach contents, leading to risks of respiratory obstruction, pulmonary inflammation and aspiration pneumonia.

While tolerance to the emetic side-effects may develop following repeated dosing, in some instances these problems may remain, requiring concurrent administration of an anti-emetic agent. Anti-histamines, anti-muscarinics and dopamine receptor antagonists have been found to be effective in combatting opioid-induced nausea and vomiting. Occasionally, 5-HT3 antagonist agents such as ondansetron and granesetron have also been used. However, treating the side-effects of one drug by the addition of another is far from an optimal solution, as the added drug may contribute its own adverse event profile, or create synergistic adverse events with the original opioid analgesic. For example, in the case of ondansetron, headaches have been reported, while dopamine antagonists such as metoclopramide may introduce other CNS disorientating side-effects.

There is clearly still a need for a pharmaceutical product capable of relieving severe pain but without the GI side effects which currently blight all the major strong opioid analgesics. The present invention addresses this and other needs.

### SUMMARY OF THE INVENTION

In one embodiment, the present invention is directed to a method for minimizing the gastrointestinal side effects associated with administration of an opioid analgesic, wherein the opioid has a derivitizable group. The method comprises orally administering an opioid prodrug or a pharmaceutically acceptable salt thereof to a subject in need thereof, wherein the opioid prodrug is comprised of an opioid analgesic covalently bonded via a carbamate bond to an amino acid or peptide of 2-9 amino acids in length, and wherein upon oral administration, the prodrug or pharmaceutically acceptable salt reduces, if not eliminates, the gastrointestinal side effects associated with oral administration of the unbound opioid analgesic. The amount of the opioid is preferably a therapeutically effective amount (e.g., an analgesic effective amount).

Another embodiment is a method of treating a disorder in a subject in need thereof with an opioid without inducing gastrointestinal side effects associated with the opioid. The method comprises orally administering an effective amount of an opioid prodrug of the present invention to the subject. The disorder may be one treatable with an opioid. For example, the disorder may be pain, such as neuropathic pain or nociceptive pain. Other specific types of pain which can be treated with the opioid prodrugs of the present invention include, but are not limited to, acute pain, chronic pain, post-operative pain, pain due to neuralgia (e.g., post herpetic neuralgia or trigeminal neuralgia), pain due to diabetic neuropathy, dental pain, pain associated with arthritis or osteoarthritis, and pain associated with cancer or its treatment.

In a further embodiment, the GI side effect associated with administration of an opioid analgesic is selected from, but is not limited to nausea, dyspepsia, post operative ileus, vomiting, constipation, or a combination of these side effects.

In one embodiment, the present invention is directed to an opioid prodrug of Formula I,

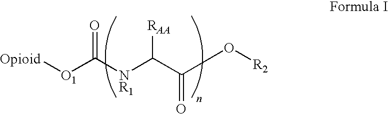

or a pharmaceutically acceptable salt thereof,

wherein,

R₁ and R₂ are independently selected from hydrogen, unsubstituted alkyl, substituted alkyl, cycloalkyl or substituted cycloalkyl group, R_{AA} is selected from a natural or non-natural amino acid side chain;

O₁ is an oxygen atom present in the unbound form of the opioid analgesic; and

n is an integer from 1 to 9 and each occurrence of R₁ and R_{AA} can be the same or different.

In another embodiment, n is 1.

In yet another embodiment, n is 2.

In yet another embodiment, n is 1 or 2 and each occurrence of R_{AA} is independently a natural amino acid side chain.

In some embodiments, n is 1, 2, 3, 4 or 5.

In a preferred embodiment, the prodrug moiety of the compound of Formula I has one, two or three amino acids (i.e., n=1, 2 or 3), while R₂ is H.

In another embodiment, the present invention is directed to a pharmaceutical composition comprising one or more of the opioid prodrugs of the present invention, and one or more pharmaceutically acceptable excipients.

In yet another embodiment, a method of reducing or eliminating pain is provided. The method comprises administering, to a subject in need thereof, an effective amount of the opioid prodrug of the present invention, or a pharmaceutical composition of the present invention.

In one embodiment, the opioid analgesic for use with the present invention (i.e., for use in a prodrug of formula I) is butorphanol, buprenorphine, codeine, dezocine, dihydrocodeine, hydrocodone, hydromorphone, hydroxymorphone, levorphanol, meptazinol, morphine, nalbuphine, oxycodone, oxymorphone, pentazocine.

In one embodiment, the

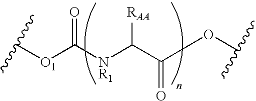

moiety of the present invention is selected from valine carbamate, L-met carbamate, 2-amino-butyric acid carbamate, ala carbamate, phe carbamate, ile carbamate, 2-amino acetic acid carbamate, leu carbamate, ala-ala carbamate, val-val carbamate, tyr-gly carbamate, val-tyr carbamate, tyr-val carbamate and val-gly carbamate.

### BRIEF DESCRIPTION OF THE FIGURES

FIG. 1 illustrates the relationship between the log concentration of oxymorphone or oxymorphone valine carbamate (expressed as the free base of oxymorphone) addition to isolated guinea pig ileum preparations and electrical field stimulation response;

FIG. 2 illustrates the relationship between the log concentration of hydromorphone or hydromorphone valine carbamate (expressed as the free base of hydromorphone) addition to isolated guinea pig ileum preparations and electrical field stimulation response;

FIG. 3 illustrates the plasma concentration of oxymorphone in dogs after either oral administration of oxymorphone itself (0.5 mg oxymorphone free base/kg) or its carbamate linked valine prodrug (0.5 mg oxymorphone free base/kg);

FIG. 4 illustrates the plasma concentration of hydromoprhone in minipigs after either oral administration of hydromorphone itself (1.0 mg hydromorphone morphone free base/kg) or its carbamate linked valine prodrug (1.0 mg hydromorphone free base/kg);

FIG. 5 illustrates the plasma concentration of meptazinol in dogs after either oral administration of meptazinol itself (1 mg meptazinol free base/kg) or its carbamate linked valine prodrug (1.0 mg meptazinol free base/kg).

FIG. 6 illustrates the plasma concentration of buprenorphine in dogs after either oral administration of buprenorphine itself (0.5 mg buprenorphine free base/kg) or its carbamate linked valine prodrug (0.5 mg buprenorphine free base/kg);

FIG. 7 illustrates the plasma concentration of oxymorphone in rats after oral administration of oxymorphone itself (2.5 mg oxymorphone free base/kg); and

FIG. 8 illustrates the plasma concentration of oxymorphone in rats after oral administration of oxymorphone valine carbamate (2.5 mg oxymorphone free base/kg).

### DETAILED DESCRIPTION OF THE INVENTION

### Definitions

As used herein:

The term “peptide” refers to an amino acid chain consisting of 2 to 9 amino acids, unless otherwise specified. In preferred embodiments, the peptide used in the present invention is 2 or 3 amino acids in length.

The term “amino acid” refers both to naturally occurring and non-naturally occurring amino acids, and carbamate derivatives thereof.

A “natural amino acid” is one of the twenty amino acids used for protein biosynthesis as well as other amino acids which can be incorporated into proteins during translation (including pyrrolysine and selenocysteine). A natural amino acid generally has the formula

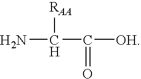

R_{AA} is referred to as the amino acid side chain, or in the case of a natural amino acid, as the natural amino acid side chain. The natural amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine and histidine.

Examples of natural amino acid sidechains include hydrogen (glycine), methyl (alanine), isopropyl (valine), sec-butyl (isoleucine), —CH₂CH(CH₃)₂ (leucine), benzyl (phenylalanine), p-hydroxybenzyl (tyrosine), —CH₂OH (serine), —CH(OH)CH₃ (threonine), —CH₂-3-indoyl (tryptophan), —CH₂COOH (aspartic acid), —CH₂CH₂COOH (glutamic acid), —CH₂C(O)NH₂ (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NHC(═NH)NH₂ (arginine) and —CH₂-3-imidazoyl (histidine).

A “non-natural amino acid” is an organic compound that is not among those encoded by the standard genetic code, or incorporated into proteins during translation. Non-natural amino acids, thus, include amino acids or analogs of amino acids other than the 20 naturally-occurring amino acids and include, but are not limited to, the D-isostereomers of amino acids. Examples of non-natural amino acids include, but are not limited to: citrulline, homocitrulline, hydroxyproline, homoarginine, homoproline, ornithine, 4-amino-phenylalanine, norleucine, cyclohexylalanine, α-aminoisobutyric acid, N-methyl-alanine, N-methyl-glycine, N-methyl-glutamic acid, tert-butylglycine, α-aminobutyric acid, tert-butylalanine, α-aminoisobutyric acid, 2-aminoisobutyric acid 2-aminoindane-2-carboxylic acid, selenomethionine, lanthionine, dehydroalanine, γ-amino butyric acid, and derivatives thereof wherein the amine nitrogen has been mono- or di-alkylated.

The term “amino” refers to a —NH₂ group;

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl. For example, C₁₋₁₀ alkyl means a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl.

The term “substituted alkyl” as used herein denotes alkyl radicals wherein at least one hydrogen is replaced by one more substituents such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The term “heterocycle” refers to a stable 3- to 15-membered ring radical which consists of carbon atoms and from one to five heteroatoms selected from nitrogen, phosphorus, oxygen and sulphur.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The terms “keto” and “oxo” are synonymous and refer to the group ═O;

The term “carbonyl” refers to a group —C(═O);

The term “carboxyl” refers to a group —CO₂H and consists of a carbonyl and a hydroxyl group (More specifically, C(═O)OH);

The terms “carbamate group,” and “carbamate,” concerns the group

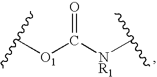

wherein the —O₁— is present in the unbound form of the opioid analgesic. Prodrug moieties described herein may be referred to based on their amino acid or peptide and the carbamate linkage. The amino acid or peptide in such a reference should be assumed to be bound via an amino terminus on the amino acid or peptide to the carbonyl linker and the opioid analgesic, unless otherwise specified.

For example, val carbamate (valine carbamate) would have the formula

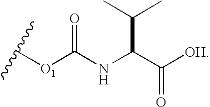

For a peptide, such as tyr-val carbamate, it should be assumed unless otherwise specified that the leftmost amino acid in the peptide is at the amino terminus of the peptide, and is bound via the carbonyl linker to the opioid analgesic to form the carbamate prodrug.

The term “carrier” refers to a diluent, excipient, and/or vehicle with which an active compound is administered. The pharmaceutical compositions of the invention may contain one or a combination of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil and sesame oil. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness) when administered to a subject. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “treating” includes: (1) preventing or delaying the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (i.e., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a subject to be treated is either statistically significant or at least perceptible to the subject or to the physician.

“Effective amount” means an amount of an opioid prodrug used in the present invention sufficient to result in the desired therapeutic response. The therapeutic response can be any response that a user or clinician will recognize as an effective response to the therapy. The therapeutic response will generally be an analgesic response affording pain relief. It is further within the skill of one of ordinary skill in the art to determine an appropriate treatment duration, appropriate doses, and any potential combination treatments, based upon an evaluation of therapeutic response.

The term “subject” includes humans and other mammals, such as domestic animals (e.g., dogs and cats).

The term “salts” can include acid addition salts or addition salts of free bases. Suitable pharmaceutically acceptable salts (for example, of the carboxyl terminus of the amino acid or peptide) include, but are not limited to, metal salts such as sodium potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; and amino acid salts such as arginate, gluconate, galacturonate, alaninate, asparginate and glutamate salts (see, for example, Berge, et al. “Pharmaceutical Salts,” J. Pharma. Sci. 1977; 66: 1).

The term “active ingredient,” unless specifically indicated, is to be understood as referring to the opioid portion of the prodrug, described herein.

### Compounds of the Invention

Without wishing to be bound to any theory, opioids may interact with the receptors within the gut wall, which can lead to adverse GI side effects (Holzer (2007). Expert Opin. Investig. Drugs 16, 181-194; Udeh and Goldman, US National Formulary 2005).

Additionally, concurrent oral administration of the locally acting (within the gut lumen) narcotic antagonist alvimopan with various opioids has been shown to markedly reduce the adverse GI effects of the latter, in terms of constipation, nausea and vomiting (Linn and Steinbrook (2007). Tech. in Regional Anaes. and Pain Mangmt 11, 27-32). Furthermore, a recently introduced combination product (Targin®) comprising oxycodone and the largely GI confined mu (μ) receptor antagonist naloxone, in a 2:1 ratio, has been shown to be associated with a reduced constipatory effect. A ˜50% reduction in the adverse effects on bowel function was reported compared with oxycodone used alone (Meissner et al. (2009). Eur. J. Pain 13, 56-64).

Therefore, without being bound to any particular theory, the prodrugs of the present invention reduce opioid induced adverse GI side effects by avoiding or minimizing interaction with opioid or other relevant receptors within the gut lumen. Subsequent to absorption, the active analgesic is regenerated (i.e., the prodrug is dissociated to form the unbound opioid analgesic) to effect the desired analgesic response. One advantage of the prodrugs of the present invention is that they eliminate the need for co-administration of medicaments to reverse the adverse GI effects of opioids such as anti-emetic agents, or narcotic antagonists such as alvimopan or naloxone.

In one embodiment, the present invention is directed to an opioid prodrug of Formula I,

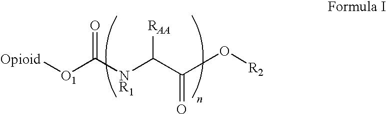

or a pharmaceutically acceptable salt thereof,

wherein,

R₁ and R₂ are independently selected from hydrogen, unsubstituted alkyl, substituted alkyl, cycloalkyl or substituted cycloalkyl group,

R_{AA} is selected from a natural or non-natural amino acid side chain;

O₁ is an oxygen atom present in the unbound form of the opioid analgesic; and

n is an integer from 1 to 9 and

each occurrence of R₁ and R_{AA} can be the same or different.

In some embodiments, n is 1, 2, 3, 4 or 5.

In a preferred embodiment, the prodrug moiety of the compound of Formula I has one, two or three amino acids (i.e., n=1, 2 or 3), while R₂ is H.

In one embodiment of the invention, an amino acid can bind to R_{AA} to create a branched peptide.

Opioid analgesics within the scope of the present invention, include, but are not limited to butorphanol, buprenorphine, codeine, dezocine, dihydrocodeine, hydrocodone, hydromorphone, hydroxymorphone, levorphanol, meptazinol, morphine, nalbuphine, oxycodone, oxymorphone, and pentazocine.

Preferred prodrug moieties (i.e., the

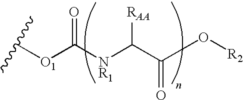

moiety) of the present invention include valine carbamate, leucine carbamate and isoleucine carbamate as single amino acid prodrug moities. Dipeptide moieties that are preferred include valine-valine carbamate, alanine-alanine carbamate and valine-glycine carbamate.

However, peptides comprising any of the naturally occurring amino acids, as well as non-natural amino acids, can be used in the present invention. Examples of non-natural amino acids are given above.

The 20 naturally occurring amino acids used for protein biosynthesis, as well as their abbreviations, are given in Table 1 below.

TABLE 1
Natural Amino acids (used for protein
biosynthesis) and Their Abbreviations

| Amino acid | 3 letter code | 1-letter code |
|---|---|---|
| Alanine | ALA | A |
| Cysteine | CYS | C |
| Aspartic Acid | ASP | D |
| Glutamic Acid | GLU | E |
| Phenylalanine | PHE | F |
| Glycine | GLY | G |
| Histidine | HIS | H |
| Isoleucine | ILE | I |
| Lysine | LYS | K |
| Leucine | LEU | L |
| Methionine | MET | M |
| Asparagine | ASN | N |
| Proline | PRO | P |
| Glutamine | GLN | Q |
| Arginine | ARG | R |
| Serine | SER | S |
| Threonine | THR | T |
| Valine | VAL | V |
| Tryptophan | TRP | W |
| Tyrosine | TYR | Y |

The amino acids employed in the opioid prodrugs for use with the present invention are preferably in the L configuration (i.e., have a negative optical rotation). The present invention also contemplates prodrugs of the invention comprised of amino acids in the D configuration, or mixtures of amino acids in the D and L configurations.

In another embodiment, the prodrug peptide moiety comprises a single amino acid, and when bound to the opioid analgesic, can be alanine carbamate, 2-amino-butyric acid carbamate, L-methionine carbamate, valine carbamate, or 2-amino acetic acid carbamate.

In other embodiments, the prodrug of the present invention comprises a dipeptide moiety, and can be tyrosine-valine carbamate, tyrosine-glycine-carbamate or valine-tyrosine carbamate.

The opioid analgesic of the present invention is conjugated to a peptide (which can be a single amino acid) through a carbamate linkage to the N-terminus of the peptide or amino acid. The peptide or amino acid can be conjugated to any free oxygen on the opioid analgesic.

In one embodiment, the peptide/amino acid (or multiple peptides or amino acids) can be bound to one of two (or both) possible locations in the opioid molecule. For example, morphine and dihydromorphine have hydroxyl groups at carbon 3 and carbon 6. A peptide or amino acid can be bound at either, or both of these positions. Carbamate linkages can be formed at either site, and upon peptide cleavage, the opioid will revert back to its original form. This general process is shown below in scheme 1, for three types of morphine prodrugs (i.e., with a peptide or amino acid linked at either or both the third and sixth carbons). For scheme 1, R₁, R₂ and R_{AA} are defined above, as provided for Formula I.

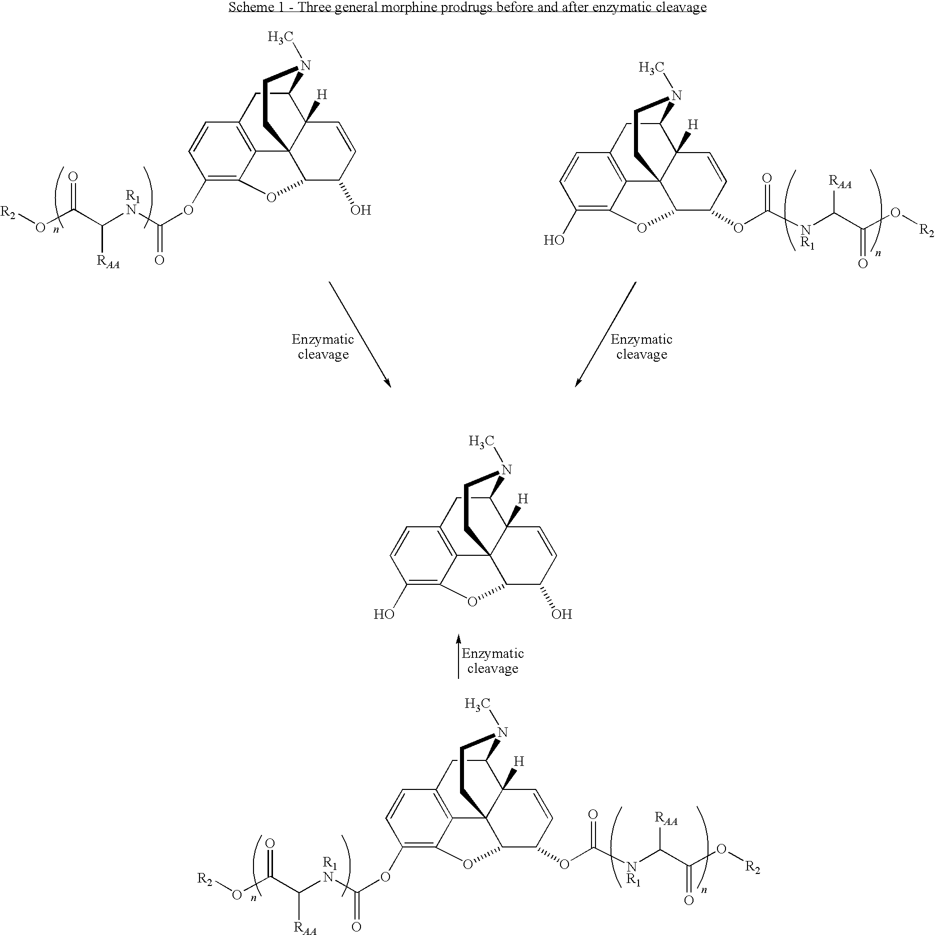

When a ketone is present in the opioid scaffold (e.g., the ketone at the 6 position of hydromorphone, and oxycodone), the ketone can be converted to its corresponding enolate and reacted with a modified peptide reactant (which can be a modified amino acid, see Examples) to form a prodrug. This linkage is depicted below in scheme 2, using hydromorphone as an example. Upon peptide cleavage, the prodrug will revert back to the original hydromorphone molecule, with the keto group present. Oxycodone can also have a peptide or amino acid linked at the 14 position, where a hydroxyl group is present. An oxycodone prodrug with a carbamate linkage at position 14 is shown in scheme 3, below. Additionally, the ketone in oxycodone can be converted to its corresponding enolate and reacted with a modified peptide reactant (which can be a modified amino acid, see Examples) to form a prodrug (not shown). For schemes 1-3, R₁, R₂, R_{AA} and n are defined as provided for Formula I.

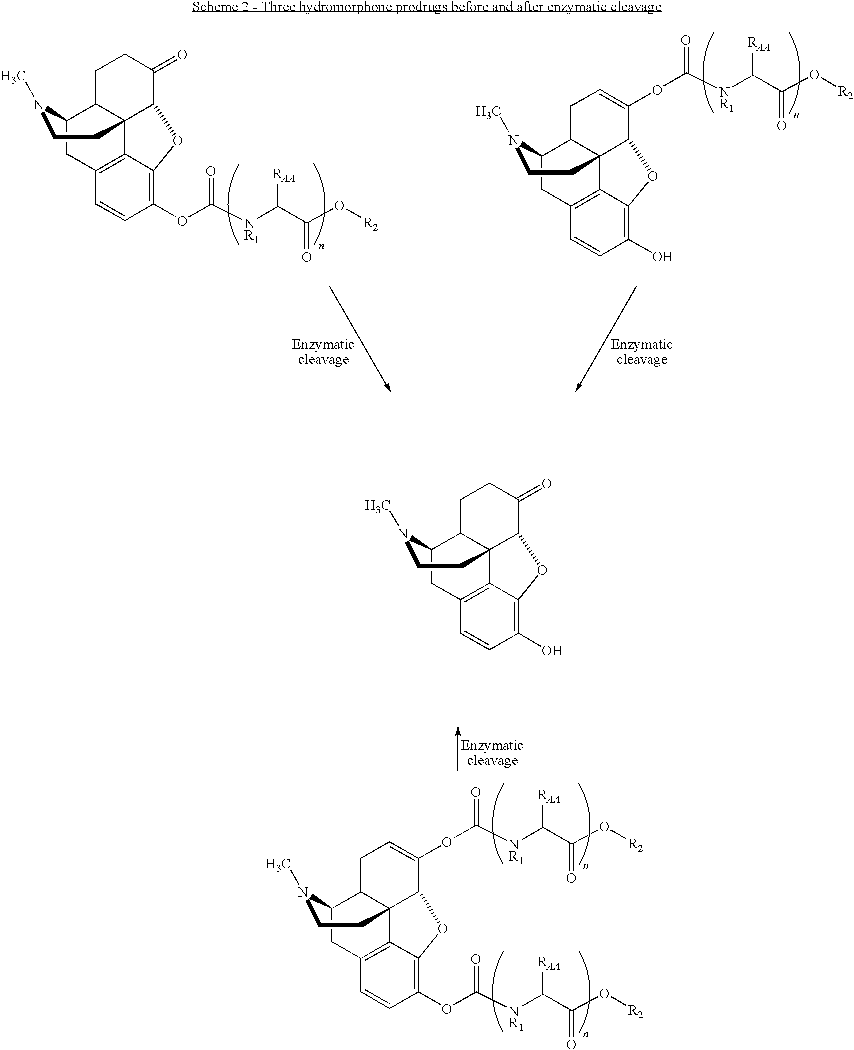

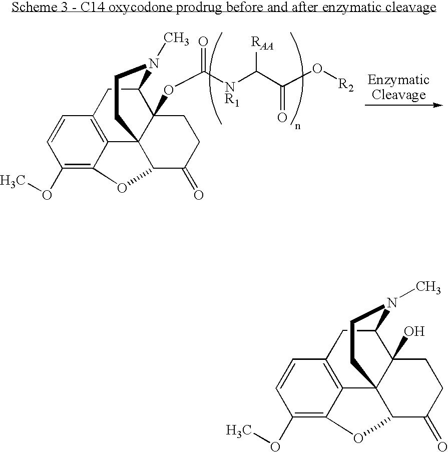

### Oxymorphone Prodrugs of the Present Invention

In one embodiment, prodrugs of the present invention are directed to novel oxymorphone prodrugs of Formula II, below.

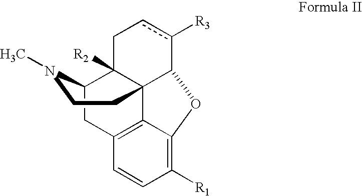

or a pharmaceutically acceptable salt thereof,

wherein,

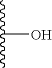

R₁ and R₂ are independently selected from and

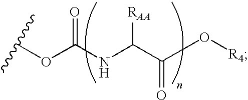

the dashed line in Formula II is absent when R₃ is

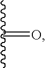

and a bond when R₃ is not

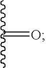

R₃ is selected from

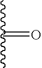

and;

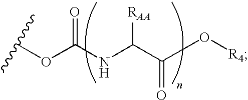

each occurrence of R₄ is independently selected from hydrogen, a substituted alkyl group and an unsubstituted alkyl group;

R_{AA} is selected from a natural or non-natural amino acid side chain and each occurrence of R_{AA} can be the same or different;

n is an integer selected from 1 to 9 and each occurrence of n can be the same or different;

and at least one of R₁, R₂, and R₃ is

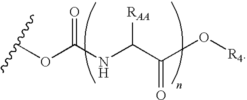

In one embodiments, n is 1, 2 or 3.

In a preferred embodiment, n is 1, 2 or 3 and R₄ is H.

In another embodiment, n is 1.

In yet another embodiment, n is 2.

In yet another embodiment, n is 1 or 2 and each occurrence of R_{AA} is independently a natural amino acid side chain.

In one embodiment, oxymorphone prodrugs of Formulae III-VI are provided. For Formulae III-VI, R₄, R_{AA} and n are defined as for Formula II. In one embodiment, n is either 1, 2, 3 or 4 and R₄ is H. Each occurrence of R_{AA} and n can be the same or different.

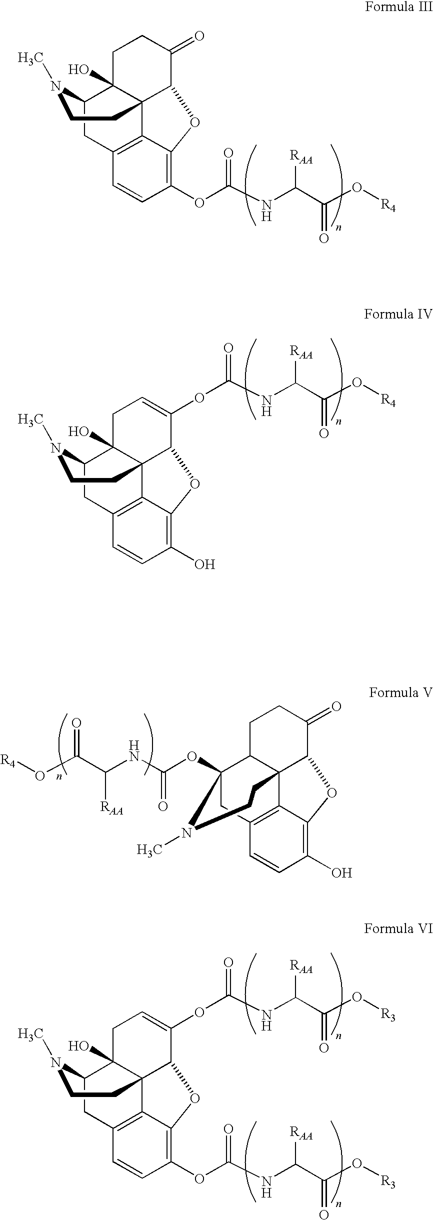

A preferred embodiment of the oxymorphone carbamate prodrug of Formula II is a prodrug wherein the amino acid side chain comprises a non-polar or an aliphatic amino acid, including the single amino acid prodrug oxymorphone valine carbamate, shown below.

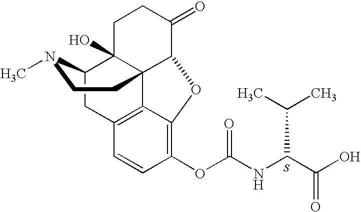

### Oxymorphone Valine Carbamate

In one embodiment, the invention is directed to the following oxymorphone and carbamate prodrugs—oxymorphone-S-ile carbamate, oxymorphone-S-leu carbamate, oxymorphone-S-asp carbamate, oxymorphone-S-met carbamate, oxymorphone-S-his carbamate, oxymorphone-S-phe carbamate, oxymorphone-S-ser carbamate.

In a preferred oxymorphone dipeptide embodiment (i.e., n is 2), the compound is selected from oxymorphone-S-val-val carbamate, oxymorphone-S-ile-ile carbamate and oxymorphone-S-leu-leu carbamate.

### Hydromorphone Prodrugs of the Present Invention

In one embodiment, the present invention is directed to a hydromorphone prodrug of Formula VII,

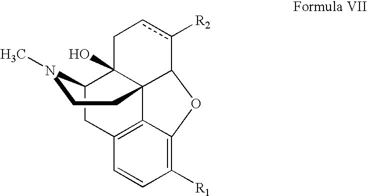

or a pharmaceutically acceptable salt thereof,

wherein,

R₁ is selected from

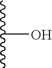

and;

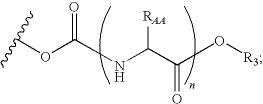

R₂ is selected from

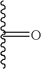

and;

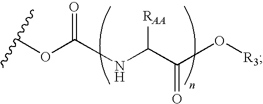

the dashed line in Formula VII is absent when R₂ is

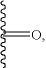

and a bond when R₂ is not

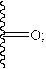

each occurrence of R₃ is independently selected from hydrogen, a substituted alkyl group and an unsubstituted alkyl group;

R_{AA} is selected from a natural or non-natural amino acid side chain and each occurrence of R_{AA} can be the same or different;

n is an integer selected from 1 to 9 and each occurrence of n can be the same or different;

with the proviso that when R₂ is

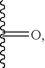

### R₁ is not

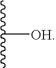

In some embodiments, n is 1, 2 or 3.

In a preferred embodiment, n is 1, 2 or 3 and R₃ is H.

In another embodiment, n is 1.

In yet another embodiment, n is 2.

In yet another embodiment, n is 1 or 2 and each occurrence of R_{AA} is independently a natural amino acid side chain.

In one embodiment, the present invention is directed to hydromorphone prodrugs of Formulae VIII-X. Hydromorphone dipeptide prodrugs (i.e., hydromorphone is derivatized with two prodrug moieties—either two amino acids, two peptides, or one amino acid and one peptide), is shown below in Formula X. For Formulae VIII-X, R₃, R_{AA} and n are defined in the same manner as defined for Formula VII. Each occurrence of R_{AA} and n can be the same or different. In a preferred embodiment for formulae VIII-X, n is 1, 2, 3 or 4 and R₄ is H.

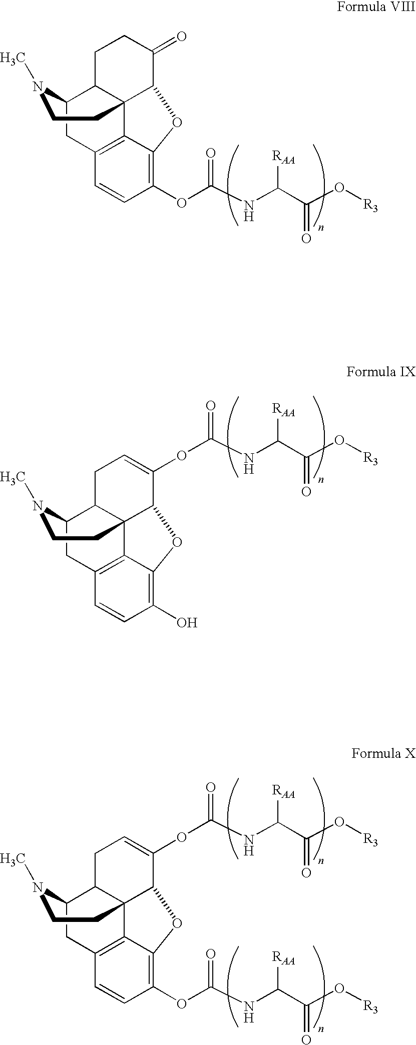

A preferred embodiment of the hydromorphone carbamate prodrug of Formula VII is a prodrug wherein the amino acid side chain comprises a non-polar or an aliphatic amino acid, including the single amino acid prodrug hydromorphone valine carbamate, shown below.

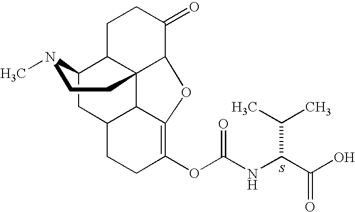

### Hydromorphone Valine Carbamate

In one embodiment, the invention is directed to the following hydromorphone carbamate prodrugs—hydromorphone-S-ile carbamate, hydromorphone-S-leu carbamate, hydromorphone-S-asp carbamate, hydromorphone-S-met carbamate, hydromorphone-S-his carbamate, hydromorphone-S-phe carbamate, and hydromorphone-S-ser carbamate.

In a preferred hydromorphone dipeptide embodiment (i.e., n is 2), the carbamate prodrug is selected from hydromorphone-S-val-val carbamate, hydromorphone-S-ile-ile carbamate and hydromorphone-S-leu-leu carbamate.

### Meptazinol Compounds of the Present Invention

The novel meptazinol compounds of the present invention include prodrugs of Formula XI:

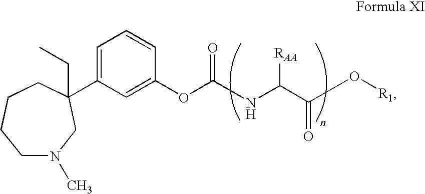

or a pharmaceutically acceptable salt thereof,

wherein,

R₁ is H, an unsubstituted alkyl group, or a substituted alkyl group,

n is an integer from 1 to 9;

R_{AA} is a natural or non-natural amino acid side chain, and each occurrence of R_{AA} can be the same or different.

In one embodiment, n is 1, 2 or 3.

In a preferred embodiment, n is 1, 2 or 3 and R₁ is H.

In another embodiment, n is 1.

In yet another embodiment, n is 2.

In yet another embodiment, n is 1 or 2 and each occurrence of R_{AA} is independently a natural amino acid side chain.

A preferred embodiment of the meptazinol prodrug of Formula XI is a prodrug wherein the amino acid side chain comprises a non-polar or an aliphatic amino acid, including the single amino acid prodrug meptazinol valine carbamate, shown below.

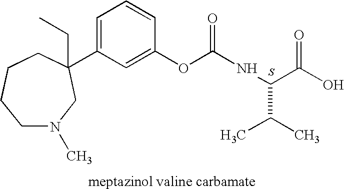

Single amino acid meptazinol carbamate prodrugs of the present invention include meptazinol-(S)-ile carbamate, meptazinol-(S)-leu carbamate, meptazinol-(S)-asp carbamate, meptazinol-(S)-met carbamate, meptazinol-(S)-his carbamate, meptazinol-(S)-phe carbamate and meptazinol-(S)-ser carbamate.

In a preferred meptazinol dipeptide embodiment (i.e., n is 2), the compound is selected from meptazinol-(S)-val-val carbamate, meptazinol-(S)-ile-ile and meptazinol-(S)-leu-leu.

Buprenorphine Prodrugs of the Present Invention

The novel buprenorphine compounds of the present invention include prodrugs of Formula XII:

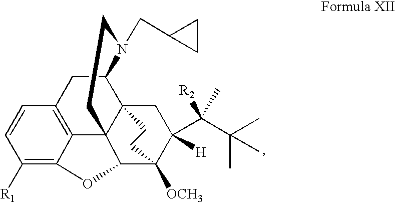

or a pharmaceutically acceptable salt thereof,

wherein,

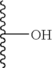

R₁ and R₂ are independently selected from and

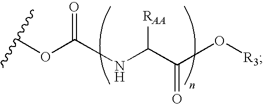

R_{AA} is selected from a natural or non-natural amino acid side chain and each occurrence of R_{AA} can be the same or different;

Each occurrence of R₃ is independently H, an unsubstituted alkyl group, or a substituted alkyl group,

and at least one of R₁ and R₂ is

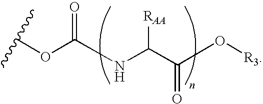

In various embodiments, n is 1, 2 or 3.

Each occurrence of “n” can be the same or different.

In preferred embodiments, n=1, 2 or 3 and R₃═H.

In another embodiment, n is 1.

In yet another embodiment, n is 2.

In yet another embodiment, n is 1 or 2 and each occurrence of R_{AA} is independently a natural amino acid side chain.

In other buprenorphine embodiments, compounds of the present invention are directed to compounds of Formulae XIII-XV, shown below. R₃, R_{AA} and n are defined in the same manner as defined for Formula XII. In a preferred embodiment for formulae XII-XV, n is 1, 2, 3 or 4 and R₄ is H. Each occurrence of R_{AA} and n can be the same or different.

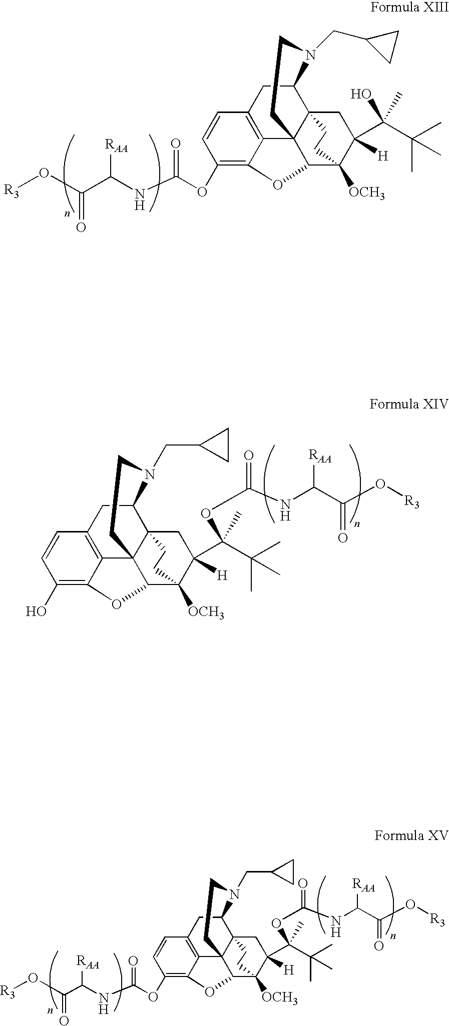

A preferred embodiment of the buprenorphine carbamate prodrug of Formula XII is a prodrug wherein the amino acid side chain comprises a non-polar or an aliphatic amino acid, including the single amino acid carbamate prodrug buprenorphine valine carbamate shown below.

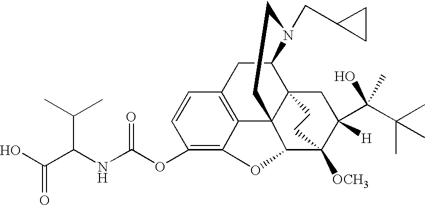

### Burprenorphine Valine Carbamate

Single amino acid burprenorphine carbamate prodrugs of the present invention include buprenorphine-(S)-ile carbamate, buprenorphine-(S)-leu carbamate, buprenorphine-(S)-asp carbamate, buprenorphine-(S)-met carbamate, buprenorphine-(S)-his carbamate, buprenorphine-(S)-phe carbamate; buprenorphine-(S)-ser carbamate.

In a preferred buprenorphine dipeptide embodiment (i.e., n is 2), the compound is selected from buprenorphine-(S)-val-val carbamate, buprenorphine-(S)-ile-ile and buprenorphine-(S)-leu-leu.

Preferred amino acids described throughout the specification are all in the L configuration, however, the present invention also contemplates prodrugs of Formulae I-XV comprised of amino acids in the D configuration, or mixtures of amino acids in the D and L configurations.

In one embodiment, the single amino acid and dipeptide prodrug moieties listed above are used with at least one of the following opioid analgesics, to form an opioid prodrug conjugate—butorphanol, codeine, dezocine, dihydrocodeine, hydrocodone, hydroxymorphone, levorphanol, morphine, nalbuphine, oxycodone, and pentazocine.

In one embodiment, the present invention is directed to prodrug moiety permutations drawn from valine, leucine, isoleucine, alanine and glycine. These prodrug moieties can be used with any of the opioid analgesics described herein, including, but not limited to hydromorphone, oxymorphone, buprenorphine and meptazinol. Yet further embodiments may include permutations drawn from these nonpolar aliphatic amino acids with the nonpolar aromatic amino acids, tryptophan and tyrosine.

Alternatively, non-natural amino acid may also be used as the prodrug moiety or a portion thereof. If a non-natural amino acid is used in a peptide, the peptide can include only non-natural amino acids, or a combination of natural and non-natural amino acids.

### Advantages of the Compounds of the Invention

Without wishing to be bound to any particular theory, it is believed that the amino acid or peptide portion of the opioid prodrug of the present invention selectively exploits the inherent di- and tripeptide transporter Pept1 within the digestive tract to effect absorption. It is believed that the opioid is subsequently released from the amino acid or peptide prodrug into the systemic circulation by hepatic and extrahepatic hydrolases that are, in part, present in plasma.

Furthermore, the prodrugs of the present invention temporarily inactivate the respective opioid, precluding any potential for local opioid action within the gut lumen on opioid or other receptors, thus, avoiding the adverse GI side effects such as constipation, commonly associated with opioid or other administration. Once absorbed, however, the opioid prodrug of the present invention is metabolized by plasma and liver esterases to the pharmacologically active opioid species which can then elicit its centrally mediated analgesic effects.

Reduction of the adverse GI side-effects associated with opioid administration is an advantage of using a prodrug of the present invention. As stated above, oral administration of a temporarily inactivated opioid would, during the absorption process, preclude access of active drug species to the μ-opioid receptors within the gut wall. The role that these peripheral μ-opioid receptors play on gut transit has recently been demonstrated by co-administration of peripherally confined narcotic antagonists such as alvimopan, and naloxone. (Linn and Steinbrook (2007). Tech in Reg. Anaes. and Pain Management 11, 27-32). Co-administration of these active agents with normally constipating opioid analgesics such as oxycodone has shown a reduction in effects on gut transit, without adversely affecting systemically mediated analgesia. Thus, oral administration of a transiently inactivated opioid may similarly avoid such problems of locally mediated constipation, without the need for co-administration of a peripheral μ-opioid antagonist.

Another potential advantage of the use of such prodrugs is a reduced likelihood of intravenous or intranasal abuse. The propensity for intravenous (i.v.) abuse is minimized by the slower rate formation of the active principal from the prodrug and consequent attainmment of Cₘₐₓ after i.v. dosing compared to that after i.v. dosing of the drug itself. Therefore, i.v. administration of the prodrug would give a “euphoric rush” less than the opioid itself.

Intranasal abuse of these amino acid/peptide prodrugs may be reduced by their negligible absorption from the nasal mucosa. This is due to the profound differences in physicochemical properties between parent opioids and the highly water soluble amino/peptide prodrugs disclosed herein. Opioid amino acid/peptide conjugates are not to be absorbed by simple diffusion due to their high water solubility and also adverse LogP values. Instead they would rely upon active transporters, such as Pept1 to assist in absorption, which while present in the gut, are essentially absent in the nasal mucosa.

### Uses of the Invention

A method for reducing or eliminating pain with one or more opioid prodrugs of the present invention is provided. The method comprises administering to a subject in need thereof (e.g., an effective amount of) a prodrug of the present invention, or a composition of the present invention. In one embodiment, the method comprises administering to a subject in need thereof a prodrug of any of Formulae I-XV, or a composition thereof.

The types of pain that can be treated includes neuropathic pain and nociceptive pain. Other specific types of pain which can be treated with the opioid prodrugs of the present invention include, but are not limited to, acute pain, chronic pain, post-operative pain, pain due to neuralgia (e.g., post herpetic neuralgia or trigeminal neuralgia, pain due to diabetic neuropathy, dental pain, pain associated with arthritis or osteoarthritis, and pain associated with cancer or its treatment.

In the methods of treating pain, the prodrugs encompassed by the present invention may be administered in conjunction with other therapies and/or in combination with other active agents (e.g., other analgesics). For example, the prodrugs encompassed by the present invention may be administered to a subject in combination with other active agents used in the management of pain. An active agent to be administered in combination with the prodrugs encompassed by the present invention may include, for example, a drug selected from the group consisting of non-steroidal anti-inflammatory drugs (e.g., ibuprofen), anti-emetic agents (e.g., ondansetron, domerperidone, hyoscine and metoclopramide), and unabsorbed or poorly bioavailable opioid antagonists to reduce the risk of drug abuse (e.g., naloxone). In such combination therapies, the prodrugs encompassed by the present invention may be administered prior to, concurrent with, or subsequent to the other therapy and/or active agent. The prodrug and other active agent(s) may also be incorporated into a single dosage form.

Another embodiment of the invention is a method of minimizing one or more gastrointestinal side effects in a patient receiving an unbound opioid analgesic, where the gastroinstestinal side effects result from or are aggravated by the administration of the opioid analgesic. The method comprises (i) discontinuing administration of the opioid analgesic to the patient, and (ii) administering to the patient an effective amount of an opioid carbamate prodrug of the present invention. According to one preferred embodiment, the opioid carbamate prodrug includes the same opioid as the discontinued opioid analgesic. The term “unbound opioid analgesic” refers to an opioid analgesic which is not a carbamate prodrug. This method is particularly useful for reducing gastrointestinal side effect(s) resulting from or aggravated by administration of the unbound opioid analgesic for pain relief.

The present invention is directed to the use of new amino acid and peptide prodrugs of the established opioid analgesic agents and methods for decreasing gastrointestinal side-effects with the prodrugs. These prodrugs comprise carbamate linked single amino acids or short peptides, preferably from 1 to 5 amino acids in length, attached to a phenolic or hydroxylic functional group within the drug molecule. The prodrug moiety renders these compounds temporarily inactive as opioid binding agents.

Without being bound by any particular theory, it is believed that the subject receiving the prodrug will avoid, or experience reduced GI side effects (e.g., emesis, constipation) associated with opioid compounds that bind to the μ-opioid, cholinergic, or other receptors located in the gut. Once absorbed, however, such prodrugs would be metabolized by plasma and liver enzymes to the pharmacologically active opioid species which can then elicit its centrally mediated analgesic effects. However, it is to be understood that the present invention is not limited to the foregoing hypothesis, and the prodrug compounds and methods disclosed herein can act by some other mechanism to reduce or eliminate GI side effects associated with unmodified opioid analgesics.

Accordingly, the present invention provides compounds, compositions and methods for reducing the GI side effects associated with opioid analgesics that are mediated by the μ-opioid or cholinergic receptors resident in the gut.

Additionally, the invention provides compositions for, and methods of reducing gastrointestinal side effects brought on by classical opioid analgesics, as well as pain from POI.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. For highly potent agents such as buprenorphine, the daily dose requirement may, for example, range from 0.5 to 50 mg, preferably from 1 to 25 mg, and more preferably from 1 mg to 10 mg. For less potent agents such as meptazinol, the daily dose requirement may, for example, range from 1 mg to 1600 mg, preferably from 1 mg to 800 mg and more preferably from 1 mg to 400 mg.

The doses referred to throughout the specification refer to the amount of the opioid free base in the particular compound.

If oxymorphone is the opioid used in the present invention, doses can be derived from the commercially available oxymorphone products Opana®, Numorphan® and Numorphone® factoring in any differences in oral bioavailability.

Salts, Solvates, Stereoisomers, Derivatives of the Compounds Employed in the Present Invention

The methods of the present invention further encompass the use of salts, solvates, stereoisomers of the opioid prodrugs described herein, for example salts of the prodrugs of Formulae I-XV, given above.

Typically, a pharmaceutically acceptable salt of an opioid prodrug used in the practice of the present invention is prepared by reaction of the opioid prodrug with a desired acid or base as appropriate. The salt may precipitate from solution and be collected by filtration or may be recovered by evaporation of the solvent. For example, an aqueous solution of an acid such as hydrochloric acid may be added to an aqueous suspension of the opioid prodrug and the resulting mixture evaporated to dryness (lyophilized) to obtain the acid addition salt as a solid. Alternatively, the opioid prodrug may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the opioid prodrugs may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium and calcium. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

Compounds useful in the practice of the present invention may have both a basic and an acidic center and may therefore be in the form of zwitterions.

Those skilled in the art of organic chemistry will appreciate that many organic compounds can form complexes, i.e., solvates, with solvents in which they are reacted or from which they are precipitated or crystallized, e.g., hydrates with water. The salts of compounds useful in the present invention may form solvates such as hydrates useful therein. Techniques for the preparation of solvates are well known in the art (see, e.g., Brittain. Polymorphism in Pharmaceutical Solids. Marcel Decker, New York, 1999.). The compounds useful in the practice of the present invention can have one or more chiral centers and, depending on the nature of individual substituents, they can also have geometrical isomers.

Individual isomers of the opioid prodrugs described herein may be used to practice the present invention. The description or naming of a particular compound in the specification and claims is intended to include both individual enantiomers and mixtures, racemic or otherwise, thereof. Methods for the determination of stereochemistry and the resolution of stereoisomers are well-known in the art.

Pharmaceutical Compositions Comprising the Opioid Peptide Prodrug

While it is possible that, for use in the methods of the invention, the prodrug may be administered as the unadulterated substance, it is preferable to present the active ingredient in a pharmaceutical formulation, e.g., wherein the agent is in admixture with a pharmaceutically acceptable carrier selected with regard to the intended route of administration and standard pharmaceutical practice.

Therefore, in some embodiments, the present invention is directed to a composition comprising an opioid prodrug and a pharmaceutically acceptable excipient. The prodrug can be any prodrug described herein, including a prodrug of Formulae I-XV.

The formulations of the present invention can be administered from one to four times daily, depending on the dosage. The formulations of the invention may be immediate-release dosage forms, i.e. dosage forms that release the prodrug at the site of absorption immediately, or controlled-release dosage forms, i.e., dosage forms that release the prodrug over a predetermined period of time. Controlled release dosage forms may be of any conventional type, e.g., in the form of reservoir or matrix-type diffusion-controlled dosage forms; matrix, encapsulated or enteric-coated dissolution-controlled dosage forms; or osmotic dosage forms. Dosage forms of such types are disclosed, for example, in Remington, The Science and Practice of Pharmacy, 20th Edition, 2000, pp. 858-914. The formulations of the present invention can be administered from one to six times daily, depending on the dosage form and dosage.

Prodrugs of phenolic opioid analgesics which do not result in sustained plasma drugs levels due to continuous generation of the opioid analgesic from a plasma reservoir of prodrug may require formulations that provide a sustained release of the opioid analgesic. For example, formulations that offer gastroretentive or mucoretentive benefits, analogous to those used in metformin products such as Glumetz® or Gluphage XR®, may be employed. An example of the former is a drug delivery system known as Gelshield Diffussion™ Technology while an example of the latter is a so-called Acuform™ delivery system. In both cases, the concept is to retain drug in the stomach, slowing drug passage into the ileum, maximizing the period over which absorption take place and effectively prolonging plasma drug levels. Other drug delivery systems affording delayed progression along the GI tract may also be of value.

In one aspect, the present invention provides a pharmaceutical composition comprising at least one active pharmaceutical ingredient (i.e., an opioid-peptide prodrug), or a pharmaceutically acceptable derivative (e.g., a salt or solvate) thereof, and, optionally, a pharmaceutically acceptable carrier. In particular, the invention provides a pharmaceutical composition comprising a therapeutically effective amount of at least one opioid prodrug of the present invention, or a pharmaceutically acceptable derivative thereof, and, optionally, a pharmaceutically acceptable carrier.

For the methods of the invention, the prodrug employed may be used in combination with other therapies and/or active agents (e.g., other analgesics). Accordingly, the present invention provides, in a further aspect, a pharmaceutical composition comprising at least one compound useful in the practice of the present invention, or a pharmaceutically acceptable derivative thereof, a second active agent, and, optionally a pharmaceutically acceptable carrier.

For example, the prodrugs of the present invention may be administered to a subject in combination with other active agents used in the management of pain. An active agent to be administered in combination with the prodrugs encompassed by the present invention may include, for example, a drug selected from the group consisting of non-steroidal anti-inflammatory drugs (e.g., acetaminophen and ibuprofen), anti-emetic agents (e.g., ondansetron, domerperidone, hyoscine and metoclopramide), unabsorbed or poorly bioavailable opioid antagonists to reduce the risk of drug abuse (e.g., naloxone). In such combination therapies, the prodrugs encompassed by the present invention may be administered prior to, concurrent with, or subsequent to the other therapy and/or active agent.

When combined in the same formulation it will be appreciated that the two compounds must be stable and compatible with each other and the other components of the formulation. When formulated separately they may be provided in any convenient formulation, conveniently in such manner as are known for such compounds in the art.

The prodrugs used herein may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro, 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, for example see International Patent Application No. WO 02/00196 (SmithKline Beecham).

The compounds and pharmaceutical compositions of the present invention are intended to be administered orally (e.g., as a tablet, sachet, capsule, pastille, pill, boluse, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyanisole.

The pharmaceutical compositions of the invention may contain from 0.01 to 99% weight per volume of the active material.

The present invention is further illustrated by reference to the Examples below. However, it should be noted that these Examples, like the embodiments described above, are illustrative and are not to be construed as restricting the enabled scope of the invention in any way.

### EXAMPLES

### Preparation of Prodrugs Employed in the Invention

Compounds employed in the present invention and derivatives thereof may be prepared by the general methods outlined hereinafter.

Chemicals were purchased primarily from Aldrich Chemical Company, Gillingham, Dorset and Alfa Aesar, Morecambe, Lancashire, U.K. and were used without further purification. Solvents utilized were anhydrous. Gasoline employed was the fraction boiling in the range 40-60° C.

TLC was carried out using aluminum plates pre-coated with silica gel (Kieselgel 60 F₂₅₄, 0.2 mm, Merck, Darmstadt, Germany). Visualization was by UV light or KMnO₄ dip. Silica gel (‘flash’, Kieselgel 60) was used for medium pressure chromatography.

¹H NMR spectra were recorded on a Bruker Avance BVT3200 spectrometer using deuterated solvents as internal standards.

Combustion analyses were performed by Advanced Chemical and Material Analysis, Newcastle University, U.K. using a Carlo-Erba 1108 elemental analyser.

### Example 1

### Generic Route of Synthesis of Amino Acid Carbamate Conjugates of Opioids

A route to phenolic opioid prodrugs as HCl or TFA salts via amino acid tert-butyl esters (with valine as an example) is given in Scheme 4, below.

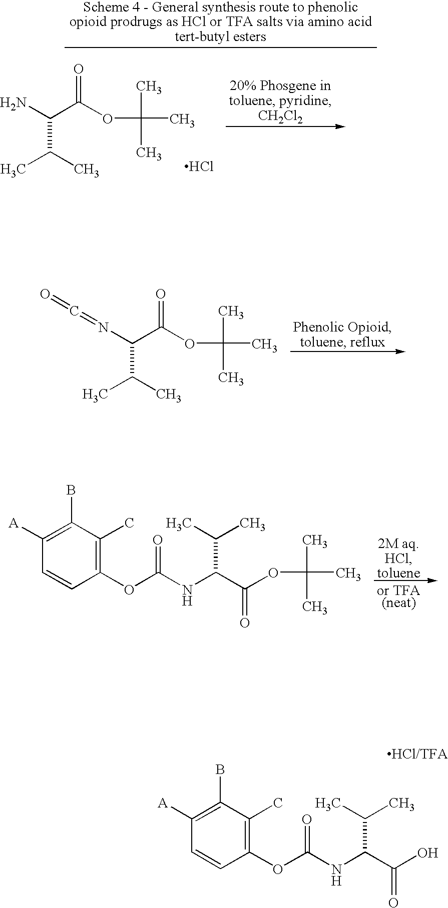

A route to phenolic opioid prodrugs via amino acid benzyl esters is given in Scheme 5, below (using valine as an example).

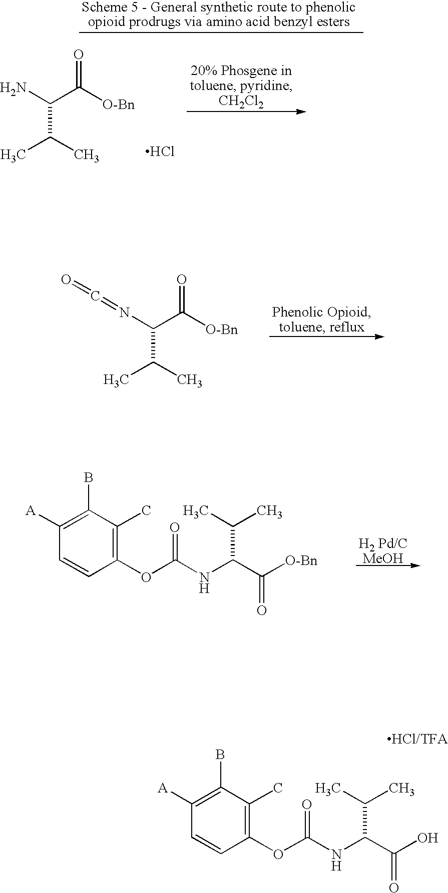

The first route (Scheme 4) is suitable for non-acid sensitive phenolic opiods, whereas the second route (Scheme 5) is suitable for those which are acid sensitive but do not contain any reducible functionalities such as double bonds.

### Example 2

### Synthesis of Oxymorphone (S)-Valine Carbamate Hydrochloride

The synthetic route is shown in scheme 6, below:

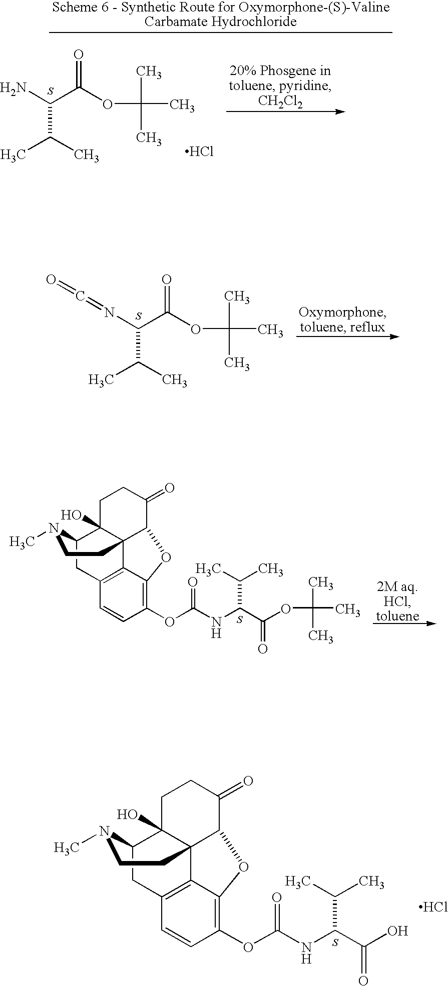

A suspension of (S)-valine tert-butyl ester hydrochloride (2.20 g, 10.5 mmol) and pyridine (3.37 mL, 42.0 mmol, 3.30 g) in anhydrous dichloromethane (60 mL) was cooled in an ice-bath under nitrogen. Next, 20% phosgene in toluene (7.35 mL, 14.0 mmol, 6.90 g) was added dropwise to the stirred mixture. Stirring was continued for a further 2 hours while the reaction was allowed to warm to room temperature. The resulting mixture was diluted with more dichloromethane, and washed with ice-cold 1 M hydrochloric acid, followed by brine and was then dried (MgSO₄) and concentrated to give an oil (2.0 g).

The oil was dissolved in anhydrous toluene (50 mL) and oxymorphone free base (1.97 g, 6.56 mmol) was added to the solution. The solution was then heated at reflux for 4 hours (the oxymorphone was not initially soluble in toluene but dissolved slowly as the reaction proceeded). The solvent was then evaporated and the residue purified by medium-pressure chromatography on silica, eluting with a solvent gradient of 4→20% methanol in dichloromethane containing 0.1% triethylamine, to afford oxymorphone-(S)-valine carbamate tert-butyl ester (2.45 g, 75%), as a glassy solid.

R_{f} 0.51 (4:1 dichloromethane-methanol plus trace of triethylamine).

A portion of this material (1.87 mg, 3.74 mmol) was dissolved in toluene (30 mL) and 2M hydrochloric acid (30 mL) was added, resulting in a two-phase mixture. The mixture was vigorously stirred and heated at 45° C. (sealed flask) overnight, and then allowed to cool. The mixture was diluted with additional toluene and water and the layers separated. The aqueous layer was washed once with diethyl ether and then freeze-dried to afford oxymorphone-(S)-valine carbamate hydrochloride (1.61 g, 90%), as an off-white powder.

¹H NMR (DMSO-d₆, 300 MHz): δ 9.40 (broad s, 1H, oxymorphone OH), 8.17 (d, J=8.4 Hz, 1H, carbamate NH), 6.95 (d, J=8.1 Hz, 1H, ArH), 6.81 (d, J=8.1 Hz, 1H, ArH), 5.14 (s, 1H, CH—O—Ar), 3.87 (dd, J=8.1, 6.0 Hz, 1H, valine α-CH), 3.76 (m, 1H, CHN), 3.45 (d, J=20.1 Hz, 1H, ½×CH₂N), 3.16-2.90 (m, 4H, benzylic CH₂+½×CH₂N+½×CH₂), 2.85 (s, 3H, CH₃N), 2.67 (m, 1H, ½×CH₂), 2.11 (m, 2H, valine, CH+½×CH₂), 1.97 (d, J=13.8 Hz, 1H, ½×CH₂), 1.48 (t, J=13.8 Hz, 2H, CH₂), 0.93 (d, J=6.9 Hz, 6H, 2×valine CH₃).

LC-MS: Single peak m/z=445.10; consistent for protonated parent ion.

### Example 3

### Synthesis of Hydromorphone (S)-Valine Carbamate Trifluoroacetate

This synthetic route is outlined in Scheme 7, below:

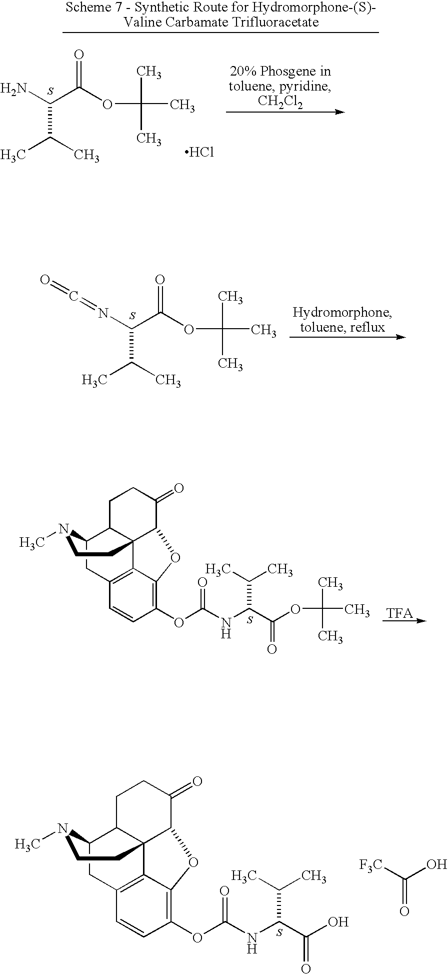

A suspension of (S)-valine tert-butyl ester hydrochloride (464 mg, 2.21 mmol) and pyridine (0.72 mL, 8.92 mmol, 0.71 g) in anhydrous dichloromethane (10 mL) was cooled in an ice-bath under nitrogen. Next, diphosgene (173 μL, 1.43 mmol, 283 mg) was added dropwise to the stirred mixture. Stirring was continued for a further 2 hours while the reaction was allowed to warm to room temperature. The resulting mixture was diluted with more dichloromethane and then washed with ice-cold 1 M hydrochloric acid, followed by brine. After which, the mixture was dried (MgSO₄) and concentrated to give an oil (0.40 g).

The oil was dissolved in anhydrous toluene (10 mL), hydromorphone free base (0.42 g, 1.47 mmol) was added and the solution. The solution was then heated at reflux for 4 hours (the hydromorphone was not initially soluble in toluene but dissolved slowly over the 2 hours as the reaction proceeded). The solvent was then evaporated and the residue purified by medium-pressure chromatography on silica, eluting with a gradient of 2→15% methanol in dichloromethane containing 0.1% triethylamine, to afford the hydromorphone-(S)-valine carbamate tert-butyl ester (0.60 g, 84%), as a glassy solid.

R_{f} 0.10 (9:1 acetone—methanol plus trace of triethylamine).

A portion of this ester (0.31 g, 0.64 mmol) was dissolved in trifluoroacetic acid (10 mL) and the resulting solution was stirred for 15 minutes. The excess solvent was evaporated and the residue azeotropically dried three times using chloroform, to afford hydromorphone-(S)-valine carbamate trifluoroacetate (340 mg, 98%), as a foam. The foam was broken up with a spatula to give a tan powder.

¹H NMR (DMSO-d₆, 300 MHz): δ 8.17 (d, J=8.4 Hz, 1H, carbamate NH), 6.94 (d, J=8.4 Hz, 1H, ArH), 6.80 (d, J=8.4 Hz, 1H, ArH), 5.15 (s, 1H, CH—O—Ar), 4.0 (b, 1H, valine α-CH), 3.88 (m, 1H, CHN), 3.35-3.20 (m, 2H, CH₂N), 3.15-3.00 (m, 1H, valine β-CH₂), 2.95-2.75 (m, 6H, CH₃N+CH₂+CH), 2.70-2.55 (m, 2H, CH₂), 2.40-2.00 (m, 3H, CH₂+CH), 1.95-1.70 (m, 2H, CH₂), 0.94 (d, J=6.6 Hz, 6H, 2×valine CH₃).

LC-MS: Single peak m/z=429.05; consistent for protonated parent ion.

### Example 4

### Synthesis of Meptazinol-(S)-Valine Carbamate

The synthetic route for meptazinol-(S)-valine carbamate is given in Scheme 8, below.

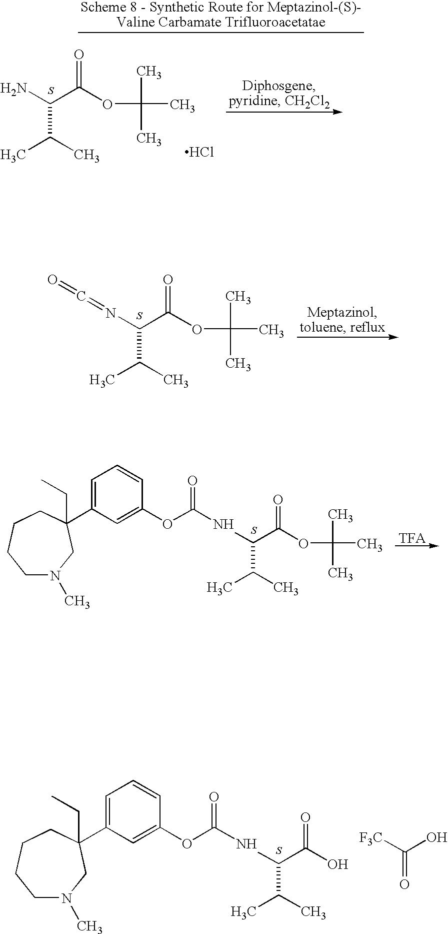

Pyridine (1.56 mL, 19.3 mmol, 1.52 g) was added to a suspension of (S)-valine tert-butyl ester hydrochloride (1.0 g, 4.77 mmol) in anhydrous dichloromethane (30 mL) under nitrogen. The mixture was stirred and cooled in an ice bath, followed by the dropwise addition of diphosgene (0.37 mL, 3.10 mmol, 0.61 g) to the reaction mixture. The reaction mixture was then allowed to warm to room temperature, while stirring was continued for 2 hours. The mixture was diluted with dichloromethane and washed with ice-cold 1M hydrochloric acid and brine. The organic layer was dried (MgSO₄) and concentrated to an oil (0.92 g).

The oil was dissolved in anhydrous toluene (40 mL). Meptazinol free base (1.05 g, 4.5 mmol) was then added, and the resulting solution was heated at reflux for 4 hours. The solvent was partially evaporated and the residue was purified by medium-pressure chromatography on silica. The resulting product was eluted with ethyl acetate containing 0.1% triethylamine, to afford 1.50 g of a viscous, colorless oil, R_{f} 0.35 (ethyl acetate-triethylamine, 99.9:0.1).

The purified material (0.75 g, 1.74 mmol) was dissolved in trifluoroacetic acid (7 mL) and the resulting solution was stirred at room temperature for 2 hours. The solution was then evaporated to dryness. Residual trifluoroacetic acid was removed by the addition of chloroform to the residue, followed by evaporation (repeated five times). The residue was dried under high vacuum at 60° C. for 3 hours to afford meptazinol-(S)-valine carbamate trifluoroacetate, as a gum.

¹H NMR (DMSO-d₆, 300 MHz): δ 8.02 (m, 1H, NH), 7.43-7.36 (m, 1H, ArH), 7.27-7.15 (m, 2H, 2×ArH), 7.06-7.00 (m, 1H, ArH), 4.01-3.89 (m, 2H, CH₂N), 3.47-3.40 (m, 1H, α-CH), 3.24-3.01 (m, 2H, CH₂N), 2.91+2.85 (m, 3H, CH₃N), 2.13 (m, 1H, β-CH), 1.95-1.40 (m, 8H, 4×CH₂), 0.96 (m, 6H, 2×isopropyl CH₃), 0.54 (m, 3H, CH₃).

LC-MS: m/z=377. Consistent for the protonated parent molecule (MH+).

### Example 5

### Synthesis of Meptazinol-(S)-Valine-(S)-Valine Carbamate

The synthetic route for meptazinol-(S)-valine-(S)-valine carbamate is shown in scheme 9, below.

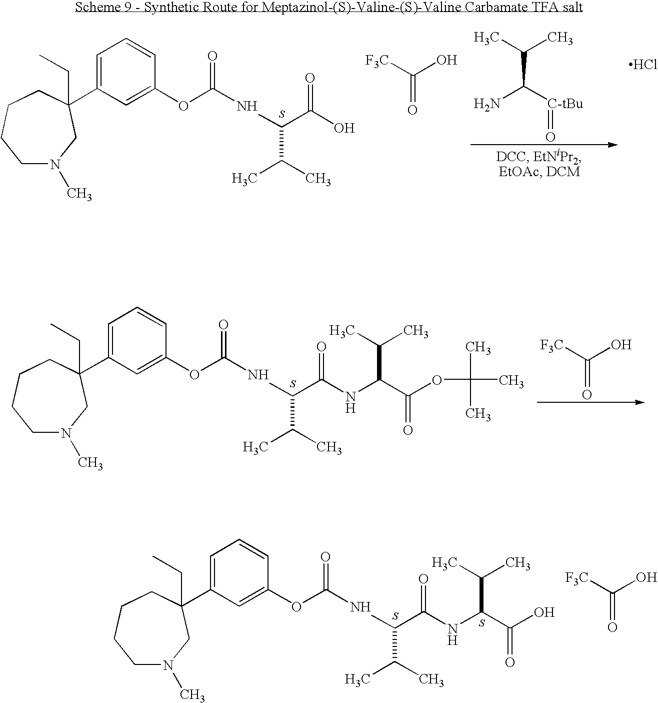

To meptazinol (S)-valine carbamate trifluoroacetate (1.30 g, 2.65 mmol), (S)-valine tert-butyl ester hydrochloride (0.67 g, 3.21 mmol) and ethyl di-isopropylamine (0.56 mL, 3.21 mmol, 0.41 g) in a stirred mixture of anhydrous dichloromethane (6 mL) and ethyl acetate (6 mL) cooled in an ice-bath under nitrogen was added dicyclohexylcarbodi-imide (0.66 g, 3.21 mmol) portionwise. The reaction mixture was allowed to warm to room temperature and stirred overnight. Ethyl acetate was added and the mixture was filtered through Celite. The solvent was evaporated and the residue was purified by medium-pressure chromatography on silica, eluting with 94.9% ethyl acetate 5% methanol-0.1% triethylamine to afford 0.75 g of a viscous, colorless oil, R_{f}0.19 (ethyl acetate-methanol, 9:1 plus trace Et₃N).

The purified material (0.75 g, 1.41 mmol) was dissolved in trifluoroacetic acid (10 mL) and the resulting solution was stirred at room temperature for 2 hours, after which the trifluoroacetic acid was evaporated. Residual trifluoroacetic acid was removed by addition of chloroform to the residue and evaporation (repeated five times). The residue was dried under high vacuum at 60° C. for 3 hours to afford meptazinol-(S)-valine-(S)-valine carbamate trifluoroacetate (0.83 g, 100%), as a viscous oil.

¹H NMR (DMSO-d₆, 300 MHz): δ 8.50 (broad s, 1H, NH), 8.00 (d, J=8.7 Hz, 1H, NH), 7.81 (d, J=8.7 Hz, 1H, ArH), 7.42-7.38 (m, 1H, ArH), 7.27-7.14 (m, 1H, ArH), 7.05-7.01 (m, 1H, ArH), 4.19-3.98 (m, 2H, CH₂N), 3.48-3.41 (m, 2H, 2×valine α-CH), ca. 3.2-3.0 (m, 2H, CH₂N), 2.90+2.84 (m, 3H, CH₃N), 2.05-1.46 (m, 10H, 4×CH₂+2×valine β-CH), 0.91 (m, 12H, 4×isopropyl CH₃), 0.52 (m, 3H, CH₃).

LC-MS: m/z=476.69. Consistent for protonated parent ion (MH⁺).

### Example 6

### Synthesis of Buprenorphine-(S)-Valine Carbamate

The synthetic route for buprenorphine-(S)-valine carbamate is given in Scheme 10, below.

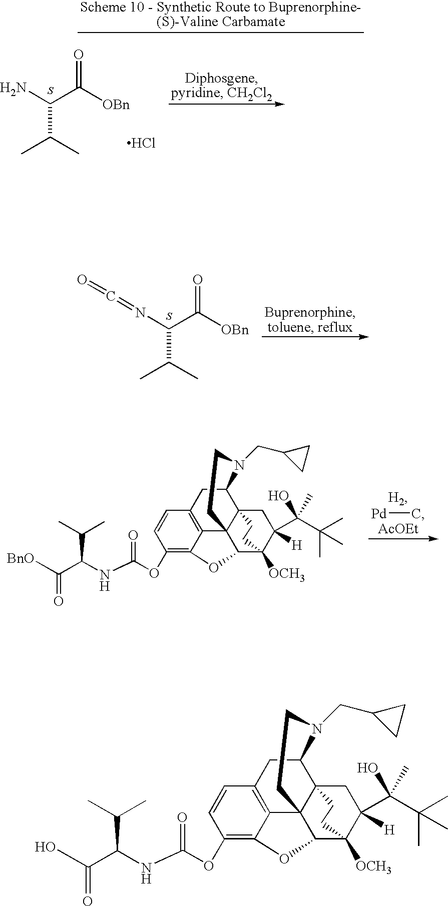

A suspension of (S)-valine benzyl ester hydrochloride (1.00 g, 2.21 mmol) and pyridine (1.34 mL, 1.29 g, 16.41 mmol) in anhydrous dichloromethane (40 mL) was cooled in an ice-bath under nitrogen. Next, diphosgene (0.32 mL, 528 mg, 2.67 mmol) was added dropwise to the stirred mixture. Stirring was continued for a further 2 hours while the reaction was allowed to warm to room temperature. The resulting mixture was diluted with more dichloromethane and then washed with ice-cold 1M hydrochloric acid, followed by brine. The mixture was dried (MgSO₄) and concentrated to give a yellow oil (0.90 g).

Buprenorphine (500 mg, 1.07 mmol) was suspended in anhydrous toluene (15 mL). A solution of (S)-valine benzyl ester isocyanate (0.75 g, 3.21 mmol) in toluene (10 mL) was added and the solution was heated at reflux overnight (the buprenorphine was not initially soluble in toluene but dissolved once the reflux temperature was achieved). The solvent was evaporated and the residue purified by medium-pressure chromatography on silica (petrol-ethyl acetate 9:1) to afford buprenorphine valine carbamate benzyl ester as a glassy solid (398 mg, 53%).

A solution of this material (398 mg, 0.64 mmol) in ethyl acetate (5 mL) was added to a suspension of 10% palladium-carbon (99 mg) in ethyl acetate (5 mL). The mixture was stirred under a hydrogen atmosphere for 5 hours and was then filtered through celite. The solvent was evaporated and the residue purified by medium-pressure chromatography on silica, eluting with a gradient of 3→10% methanol in dichloromethane, to afford buprenorphine valine carbamate (92 mg, 26%), as a white solid.

¹H NMR (DMSO-d₆, 300 MHz): δ 12.46 (br s, 1H, OH), 7.88 (d, J=8.4 Hz, 1H, NH), 6.71 (d, J=8.1 Hz, 1H, ArH), 6.50 (d, J=8.1 Hz, 1H, ArH), 6.19 (d, J=8.7 Hz, 1H, CH), 5.37 (s, 1H, CH), 4.34 (s, 1H, CH), 3.90 (m, 1H, α-H), 3.70 (m, 2H, CH), 3.27 (s, 3H, OMe), 2.86 (br d, J=18.6 Hz, 1H, CH), 2.66 (m, 1H, CH), 2.50 (br s, 1H, CH), 2.16 (m, 3H, CH), 2.08-1.82 (m, 4H, CH), 1.62-1.48 (m, 4H, CH), 1.15 (s, 3H, Methyl), 0.99 (m, 1H, CH), 0.85 (s, 9H, tert-butyl), 0.75 (m, 12H, isopropyl CH₃ and CH), 0.34 (m, 3H, 3×cyclopropyl CH), 0.00 (br s, 2H, 2×cyclopropyl CH).

LC-MS: Single peak m/z=611.20; Consistent for protonated ion.

### Example 7

### In Vitro Stability of Oxymorphone, Hydromorphone, Meptazinol and Buprenorphine Prodrugs Under Conditions Prevailing in GI Lumen

Since the GI luminal stability of these (inactive) opioid prodrugs is important if opioid-like effects on the intestinal smooth muscle are to be avoided, the rate and extent of their hydrolysis under the conditions prevailing in the GI tract was evaluated. Using USP simulated gastric and intestinal juices, the stability of oxymorphone valine carbamate, hydromorphone valine carbamate, meptazinol valine carbamate and buprenorphine valine carbamate were investigated over a 2 hour period at 37° C. Remaining drug was quantified by HPLC.

Results

All four valine carbamate prodrugs demonstrated good stability over a 2 hour period (Table 2). This period would correspond to the typical time that these prodrugs may remain in the gut before being fully absorbed. As such it is unlikely therefore that significant amounts of active opioid would come into direct contact with the gut, so minimizing any potential for direct effects of the opioid on the gut.

TABLE 2
Opioid prodrug Stability in Various Media

| | Simulated | Simulated | |
|---|---|---|---|
| | gastric fluid | intestinal fluid | Distilled water |
| | (pH 1.1): | (pH 6.8): | (pH 5.9): |
| | % remaining | % remaining | % remaining |
| Compound | after 2 h/37° C. | after 2 h/37° C. | after 2 h/20° C. |
| Oxymorphone Val | 100 | 97 | 100 |
| Carbamate | | | |
| Hydromorphine Val | 100 | 98 | 100 |
| Carbmate | | | |
| Buprenorphine Val | 100 | 98 | 100 |
| Carbamate | | | |
| Meptazinol Val | 100 | 99 | 100 |
| Carbamate | | | |

### Example 8

### In Vitro Effects of Oxymorphone and Hydromorphone and their Valine Carbamate Prodrugs on Guinea Pig Ileum Contractility

Methodology

Strips of guinea pig small intestine myenteric plexus longitudinal muscle were mounted between platinum ring electrodes. The tissue was stretched to a steady tension of about 1 g and changes in force production were recorded using sensitive transducers.

Optimal voltage for stimulation was determined while the tissue was paced with an electrical field stimulation (EFS) at 14 Hz, with a pulse width of 0.5 msec. (Trains of pulses then continued for 20 seconds, every 50 seconds).

EFS at optimal voltage continued throughout the protocol (stable responses=“baseline measurement of EFS”).

The test conditions employed were as follows:

(1) Vehicle (deionized water, added at equivalent volume additions to test articles);
(2) Hydromorphone at 6 concentrations (1 nM, 10 nM, 30 nM, 100 nM, 300 nM, 1 μM) and
(3) Hydromorphone valine carbamate at 6 concentrations (1 nM, 10 nM, 30 nM, 100 nM, 300 nM, 1 μM)
(4) Oxymorphone at 6 concentrations (1 nM, 10 nM, 30 nM, 100 nM, 300 nM, 1 μM) and
(5) Oxymorphone valine carbamate at 6 concentrations (1 nM, 10 nM, 30 nM, 100 nM, 300 nM, 1 μM)

Following 10 minutes of baseline EFS, the first addition of test article or vehicle (deionized water) was performed.

Test concentrations were added in a non-cumulative manner with PSS washes between each addition. Next, TTX (Na+ channel blocker) was added to confirm EFS responses were elicited via nerve stimulation. EFS was then stopped.

Results

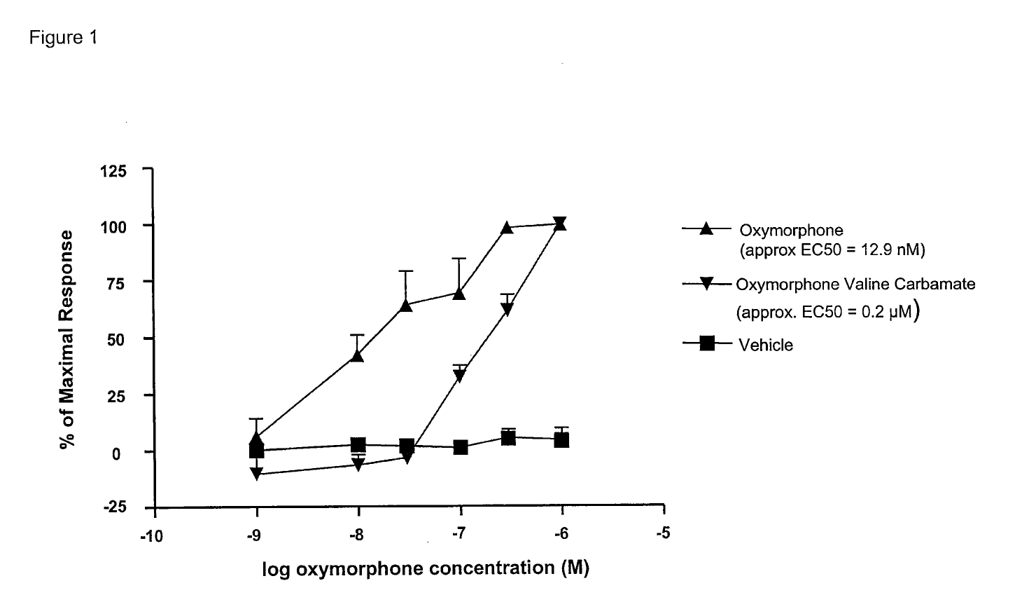

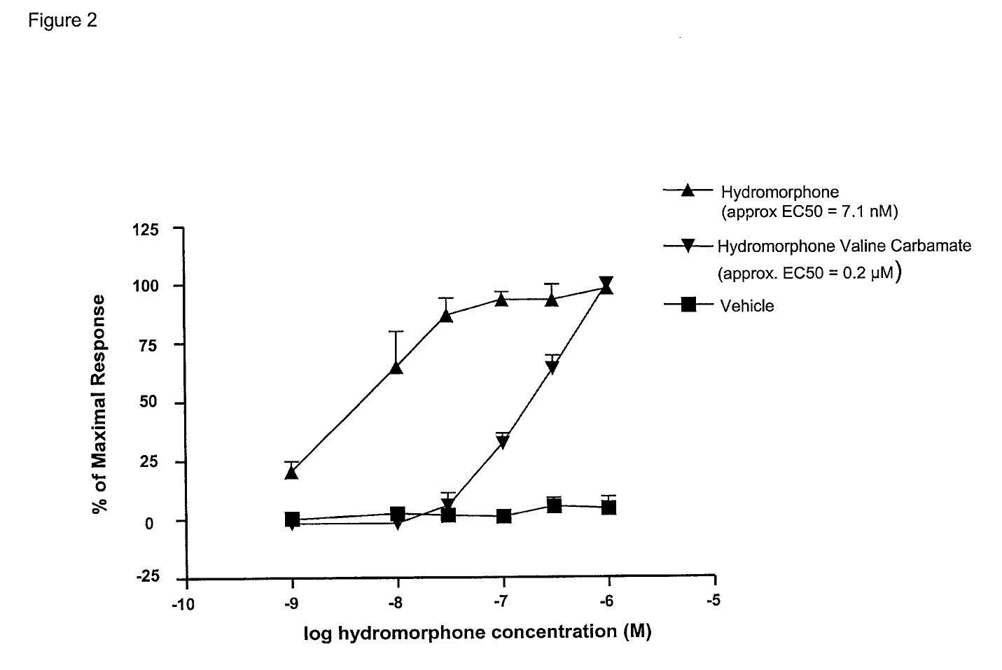

The results shown in FIGS. 1 and 2 and Table 3. These results reveal a dramatic reduction in the opioid effects of these prodrugs on ileal smooth muscle, as compared to the underivatized opioid. These results show that the prodrugs of the present invention have much less opioid mediated inhibitory effects on gut contractility, as compared to their corresponding unbound opioids.

TABLE 3
In vitro effects of oxymorphone and hydromorphone and their
valine carbamate prodrugs on guinea pig ileum contractility

| | EC₅₀ for inhibition of | | Fold |
|---|---|---|---|
| Compound | contractility | | difference |
| Oxymorphone | 12.9 | nM | 15.5 |
| Oxymorphone valine carbamate | 200 | nM | |
| Hydromorphone | 7.1 | nM | 28 |
| Hydromorphone valine carbamate | 200 | nM | |

### Example 9

### Assessment of Reduction in Intrinsic Opioid Binding Activity of buprenorphine-3-valine Carbamate

Previous examples of opioid conjugation at the 3-position with valine carbamate have consistently shown a marked reduction in opioid-like activity on guinea pig ilieum. This was seen with both oxymorphone and hydromorphone valine carbamates which showed reductions in EC₅₀ values of 15.5 and 28-fold, respectively. In the case of oxymorphone valine carbamate, the reduced opioid activity was subsequently found to directly translate into a reduced constipatory effect—as the result of precluding direct interaction with the opioid receptors in the gut.

The structural similarity of these opioid valine carbamate prodrugs with bupmoprhine valine carbamate is illustrated below. Furthermore a structurual overlay of buprenorphine valine carbamate and oxymorphone valine carbamate again emphasizes the prodrugs' close similarity in structure. In view of this, and by analogy with the reduced opioid binding activity of oxymorphone valine carbamate, buprenorphine valine carbamate will also display much less opioid binding activity in comparison with the parent drug.

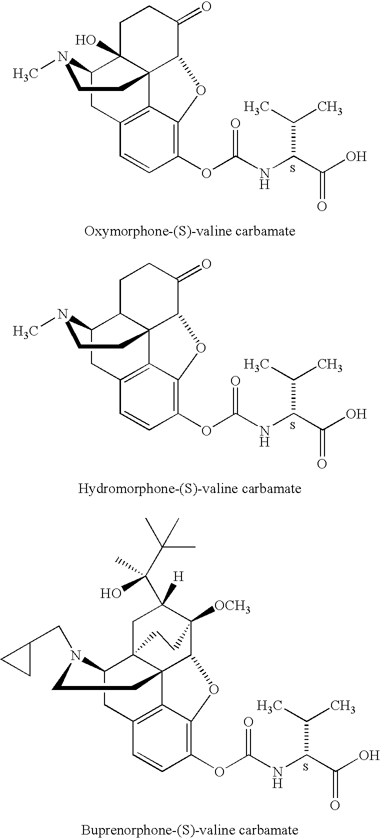

### Structural Comparability of Three Opioid Valine Carbamate Prodrugs

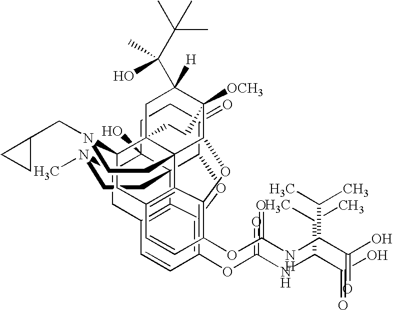

### Overlay of buprenorphine-(S)-valine carbamate (Foreground) and oxymorphone-(S)-valine Carbamate (Background)

### Example 10

### Assessment of Cholinergic Effects of Meptazinol Valine Carbamate in Isolated Gut Preparation

The direct effects of meptazinol and the meptazinol valine carbamate prodrug were assessed, using an ex vivo isolated gut smooth muscle model. Circular muscle strips of rat and human colon were dissected and set up in an organ bath system. Changes in smooth muscle force production were monitored using a pressure transducer. Nerves within the muscle strips were stimulated using an electrical field, which created paced contractions of the smooth muscle. The potential influence of these compounds on gut motility was then assessed by measuring the size of contractions. As shown in Table 3, meptazinol itself had a profound effect—reflected in the large increase in amplitude of the response to the EFS with increasing concentrations.

Examination of the data (Table 4) showing the effects of meptazinol valine carbamate on EFS induced pulsed muscle contractions showed no increase in amplitude i.e., mean force of contraction with increasing concerns suggesting this prodrug had no cholinergic effect. This is consistent with the loss of acetyl choline esterase inhibitory action found in an in vitro assessment of the meptazinol valine carbamate using a human acetyl choline in esterase preparation.

On this basis such prodrugs will not elicit the emesis seen with the parent molecule.

TABLE 4
Effects of meptazinol and meptazinol valine carbamate
on contractility of human and rat colonic strips

| | | |
|---|---|---|
| | Meptazinol | Meptazinol valine carbmate |
| Species/Compound | Mean force of | Mean force of |
| Concentration | contraction ± sd | contraction ± sd |

Rat

| | | | |
|---|---|---|---|
| 0.01 | μM | 96.87 | 104.47 |
| 0.1 | μM | 88.60 | 121.87 |
| 0.5 | μM | 85.56 | 86.04 |
| 1.0 | μM | 95.08 | 75.22 |
| 10 | μM | 190.38 | 65.57 |
| 100 | μM | 476.58 | 75.17 |

Human

| | | | |
|---|---|---|---|
| 0.01 | μM | 91.16 | 89.77 |
| 0.1 | μM | 83.34 | 83.63 |
| 0.5 | μM | 80.11 | 79.00 |
| 1.0 | μM | 82.83 | 80.56 |
| 10 | μM | 134.24 | 76.92 |
| 100 | μM | 191.75 | 79.25 |

### Example 11

### Demonstration of In Vivo Bioavailability of Opioids from their Amino Acid Prodrugs in Dogs or Minipigs

Test substances (i.e., opioid and selected prodrugs) were administered by oral gavage to a group of five dogs or minipigs in a crossover design. The characteristics of the test animals are set out in Table 5, below.

TABLE 5
Characteristics of experimental animals used in study

| | |
|---|---|
| Species | Dog (oxymorphone, buprenorphine, meptazinol) |
| | or Minipigs (hydromorphone) |
| Type | Beagle dogs or Gottingen minpigs |
| Number and sex | 5 males |
| Approximate age | 3-4 months at the start of treatment |
| Approx. bodyweight | 7-9 kg at the start of treatment |
| Source | Huntingdon Life Sciences stock |

Blood samples were taken at various times after administration and submitted to analysis for the parent drug and pro-drug using a validated LC-MS-MS assay. Pharmacokinetic parameters derived from the plasma analytical data were determined using Win Nonlin. The results are given in Table 6.

TABLE 6
Comparative pharmacokinetics of opioid drugs arising
from administration of either the drug itself or prodrugs
given in equimolar oral doses to dogs (1 mg/kg)

| | Cmax | AUC | | |
|---|---|---|---|---|
| | (mean ng/ | (mean ngh/ | Tmax | T½ |
| Compound name | mL ± SD) | mL ± SD) | (h) | (h) |
| Oxymorphone HCl | 3.80 ± 0.77 | 12.2 ± 1.60 | 0.5 | 2.2 |
| Oxymorphone valine | 21.6 ± 7.2 | 94.4 ± 33.3 | 1.5 | 2.1 |
| carbamate HCl | | | | |
| Hydromorphone | 4.55 ± 2.40 | 17.94 ± 10.46 | 1.0 | 2.6 |
| Hydromorphone valine | 4.81 ± 2.25 | 17.7 ± 8.9 | 3.0 | 1.3 |
| carbamate | | | | |
| Meptazinol | 2.57 ± 0.63 | 12.8 ± 3.4 | 0.5 | 2.8 |
| Meptazinol valine | 24.8 ± 1.4 | 67.6 ± 9.4 | 1.0 | 2.2 |
| carbamate | | | | |
| Buprenorphine | 2.88 ± 1.58 | 3.19 ± 1.63 | 0.5 | 1.2 |
| Buprenorphine valine | 3.1 ± 0.67 | 23.2 ± 11.3 | 1.0 | 5.6 |
| carbamate | | | | |

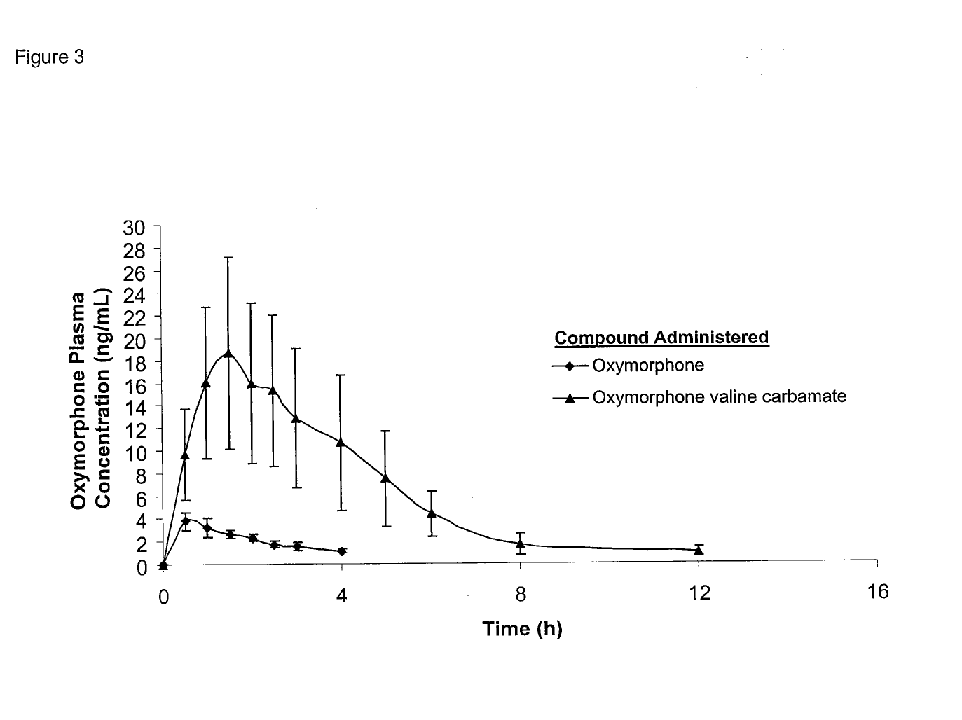

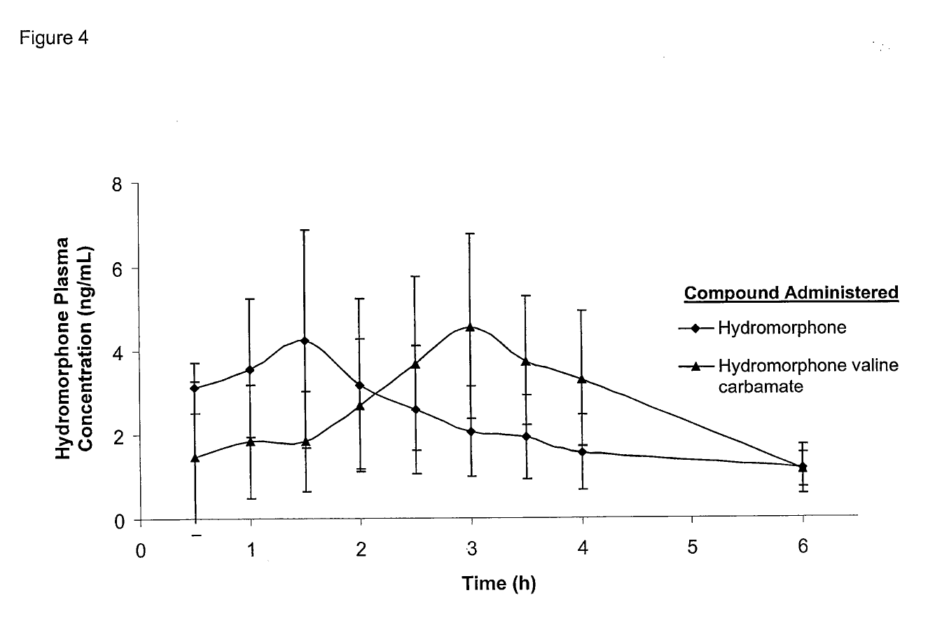

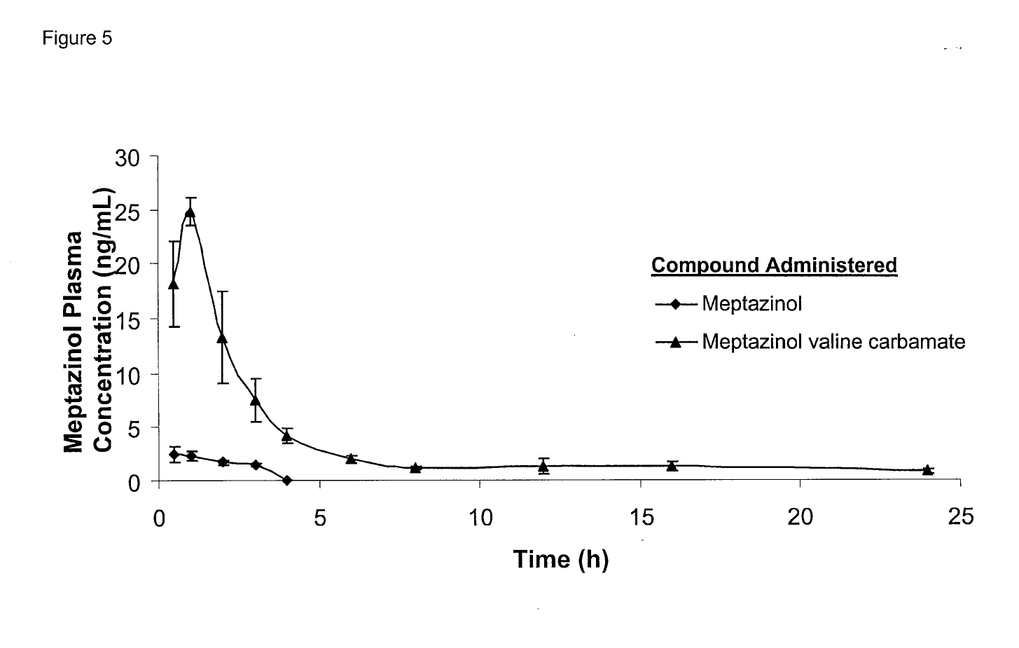

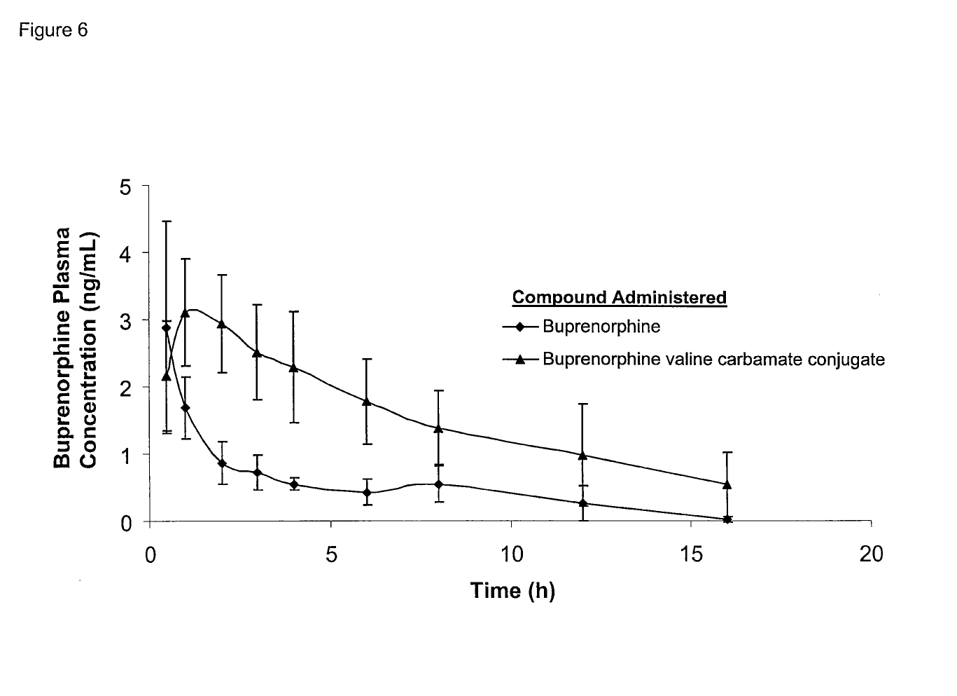

This tabulation (Table 6) of the PK parameters and FIGS. 3-6 show that after administration of these prodrugs, plasma concentrations of the active drug were at least as great as those seen after giving the drug itself. In the case of oxymorphone and meptazinol, an increase in bioavailability was evident.

### Example 12

### In Vivo Effects of Oxymorphone Valine Carbamate Versus Oxymorphone on Put Motility in the Rat

Methodology

The effects of the oxymorphone and its valine carbamate prodrug on gastro-intestinal motility in the rat were assessed by means of the charcoal propulsion test. Test treatments were administered to groups of 10 rats fasted overnight prior to the test.

The method used was based on that described by Takemori et al. (1969). J. Pharmacol. Exp. Ther., 169, 39. Test treatments were administered orally 60 minutes prior to an oral dose of 2.0 ml of a 10% suspension of charcoal in 5% gum arabic. Thirty minutes after dosing with charcoal, the rats were sacrificed and the entire gastro-intestinal tract quickly and carefully removed. The distance the charcoal meal had traveled from the pyloric sphincter towards the caecum was measured and expressed as a percentage of both the total gut length and the length of the small intestine.

Results

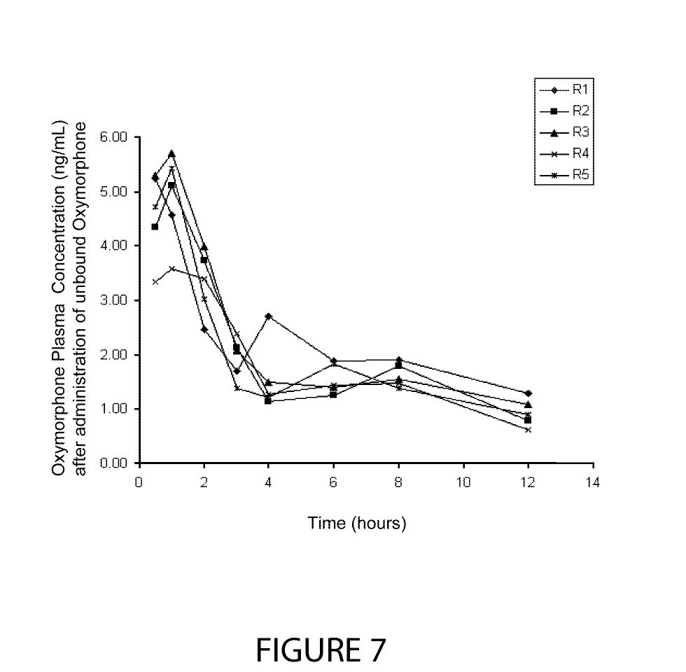

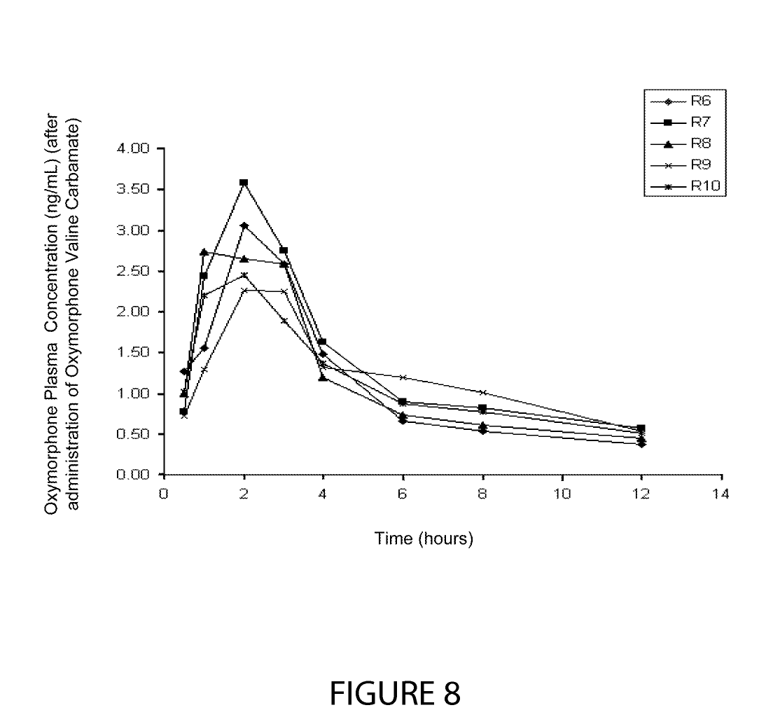

The results presented in Table 7, below, show that oxymorphone itself elicited a profound effect on gut motility delaying the passage of the charcoal meal after a 30 mg/kg dose by 70%. By contrast, oxymorphone valine carbamate delayed gut motility by only 14% after an equimolar dose. Additionally, comparative pharmacokinetic studies in the rat showed similar systemic exposure to oxymorphone whether the drug was given per se or as the prodrug (FIGS. 7 and 8, R_signifies rat no.). Thus, the lesser constipating effects of oxymorphone valine carbamate could not be simply attributed to reduced systemic exposure in the rat. These data are evidence that the valine carbamate of oxymorphone is less constipating in man than is the parent drug molecule.

TABLE 7
Effects of oral administration of oxymorphone and oxymorphone
valine carbamate on gastrointestinal motility in the rat

| | | Group mean distance | | | |
|---|---|---|---|---|---|
| | | travelled by charcoal as % of | | % change | |
| | | (±sd) | | from vehicle-treated animals | |
| | Dose | Small | Total gut | Small | Total gut |
| Oral treatment | (mg/kg) | intestine | length | intestine | length |
| Vehicle | — | 72.1 ± 8.25 | 61.3 ± 6.98 | — | — |
| (Sterile water) | | | | | |
| Oxymorphone HCl | 1 | 64.2* ± 5.05 | 54.6* ± 4.34 | −5.0 | −4.5 |
| Oxymorphone HCl | 3 | 61.2** ± 6.69 | 51.8** ± 5.70 | −9.5 | −9.4 |
| Oxymorphone HCl | 10 | 54.7** ± 10.26 | 46.5** ± 9.00 | −24.1 | −24.1 |
| Oxymorphone HCl | 30 | 20.6** ± 6.17 | 17.3** ± 5.36 | −69.5 | −69.8 |
| Oxymorphone valine | 10 | 68.5 ± 7.85 | 58.9 ± 6.95 | −4.9 | −3.9 |
| carbamate | | | | | |
| Oxymorphone valine | 30 | 62.2** ± 6.83 | 53.3* ± 5.91 | −13.7 | −13.1 |
| carbamate | | | | | |

sd Standard deviation
Statistical significance of difference from vehicle-treated group:
*p < 0.05,
**p < 0.01

### Example 13

### Assessment of Emesis Induced by Meptazinol and Meptazinol Valine Carbamate in the Ferret

Female ferrets, starved overnight, were pre-treated the following morning with naloxone by subcutaneous injection (0.5 mg/kg) using a dose volume of 1 mL/kg. This was administered to minimize the otherwise profound CNS depression seen at these relatively high doses of meptazinol. Approximately 15 minutes later the animals received, by oral gavage, either an aqueous solution of meptazinol HCl or meptazinol valine carbamate HCl using a constant dose volume of 5 mL/kg. The animals were continuously observed for 2 hours post oral treatment and any incidences of retching and vomiting were recorded.

Results

As shown in Table 8, not all of the 8 animals treated with meptazinol exhibited retching and emesis. However, those which did (50%) demonstrated marked effects with between 5 and 66 retches observed in a 2 hour period, and between 1 and 17 episodes of vomiting. By contrast, in the group of five animals treated with a comparable molar dose of meptazinol valine carbamate, only one demonstrated any retching and vomiting at all. Furthermore, this behaviour was very mild with just 2 retches and 1 vomit compared to 66 and 17, respectively, when the same animal was treated with meptazinol itself.

These emesis data provide evidence that meptazinol valine carbamate has a much lower potential to bring about this effect than does meptazinol itself, most likely due to avoiding direct contact between active drug & the stomach wall.

TABLE 8
Summary of effects of meptazinol vs meptazinol valine
carbamate on retching and vomiting in the ferret

| | | | Total number of individual | |
|---|---|---|---|---|
| Subcutaneous | Oral | Animal | incidences of: | |
| pretreatment | treatment | no. | Retching | Vomiting |
| Naloxone | Meptazinol | 2 | 0 | 0 |
| 0.5 mg/kg | HCl | 11 | 0 | 0 |
| | 150 mg/kg | 10 | 11 | 12 |
| | | 13 | 27 | 4 |
| | | 15 | 0 | 0 |
| | | 9 | 66 | 17 |
| | | 12 | 0 | 0 |
| | | 14 | 5 | 1 |
| Naloxone | Meptazinol | 4 | 0 | 0 |
| 0.5 mg/kg | valine | 9 | 2 | 1 |
| | carbamate | 11 | 0 | 0 |
| | 150 mg/kg | 14 | 0 | 0 |
| | | 13 | 0 | 0 |

Patents, patent applications, publications, product descriptions, and protocols which are cited throughout this application are incorporated herein by reference in their entireties.

The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A method of treating a disorder with an opioid without inducing GI side effects associated with the opioid, comprising orally administering an opioid prodrug or pharmaceutically acceptable salt thereof to the subject, wherein the opioid prodrug is comprised of an opioid analgesic covalently bonded through a carbamate linkage to an amino acid or peptide of 2-9 amino acids in length.

2. The method of claim 1, wherein the disorder is one treatable with an opioid.

3. The method of claim 1, wherein the opioid analgesic is selected from the group consisting of butorphanol, buprenorphine, codeine, dezocine, dihydrocodeine, hydrocodone, hydromorphone, hydroxymorphone, levorphanol, meptazinol, morphine, nalbuphine, oxycodone, oxymorphone and pentazocine.

4. The method of claim 1, wherein the amino acid or peptide is valine carbamate, L-methionine carbamate, 2-amino-butyric acid carbamate, alanine carbamate, phenylalanine carbamate, isoleucine carbamate, 2-amino acetic acid carbamate, leucine carbamate, isoleucine carbamate, valine-valine carbamate, tyrosine-glycine-carbamate, valine-tyrosine carbamate, tyrosine-valine carbamate, or valine-glycine carbamate.

5. The method of claim 1, wherein the gastrointestinal side effect is nausea, dyspepsia, post operative ileus, vomiting, gastric ulceration, diarrhea, constipation and a combination of these side effects.

6. A method of treating pain with an opioid without inducing GI side effects associated with the opioid, comprising orally administering an opioid prodrug or pharmaceutically acceptable salt thereof to the subject, wherein the opioid prodrug is comprised of an opioid analgesic covalently bonded through a carbamate linkage to an amino acid or peptide of 2-9 amino acids in length.

7. The method of claim 6, wherein the pain is nociceptive pain.

8. The method of claim 6, wherein the pain is neuropathic pain

9. The method of claim 6, wherein the opioid is meptazinol.

10. The method of claim 6, wherein the opioid is hydromorphone.

11. The method of claim 6, wherein the opioid is oxymorphone.

12. The method of claim 6, wherein the opioid is buprenorphine.

13. A method for minimizing gastrointestinal side effects in a subject in need thereof, the gastrointestinal side effects associated with the administration of an opioid analgesic, comprising: orally administering an opioid prodrug or pharmaceutically acceptable salt thereof to the subject, wherein the opioid prodrug is comprised of an opioid analgesic covalently bonded through a carbamate linkage to an amino acid or peptide of 2-9 amino acids in length, and wherein upon oral administration, the prodrug or pharmaceutically acceptable salt ameliorates at least one gastrointestinal side effect associated with oral administration of the opioid analgesic alone.

14. A method of minimizing one or more gastrointestinal side effects in a subject receiving an unbound opioid, wherein the gastroinstestinal side effects result from or are aggravated by the administration of the unbound opioid, the method comprising:
(i) discontinuing administration of the unbound opioid to the patient, and
(ii) administering an effective amount of an opioid prodrug or pharmaceutically acceptable salt thereof to the subject, wherein the opioid prodrug is comprised of an opioid covalently bonded through a carbamate linkage to an amino acid or peptide of 2 to 9 amino acids in length.

15. The method of claim 14, wherein the opioid in the carbamate prodrug is the same opioid as the discontinued opioid analgesic.

16. A compound of the formula: 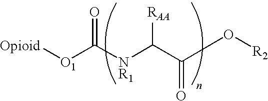
oo a pharmaceutically acceptable salt thereof,
wherein,
R₁ and R₂ are independently selected from hydrogen, unsubstituted alkyl, substituted alkyl, cycloalkyl or substituted cycloalkyl group,
R_{AA} is selected from a natural or non-natural amino acid side chain;
O₁ is an oxygen atom present in the unbound form of the opioid; and
n is an integer from 1 to 9 and each occurrence of R₁ and R_{AA} can be the same or different.

17. The compound of claim 16, wherein the opioid is selected from the group consisting of oxymorphone, hydromorphone, meptazinol and buprenorphine.

18. An opioid analgesic carbamate prodrug selected from the group consisting of hydromorphone valine carbamate, oxymorphone valine carbamate, buprenorphine valine carbamate and meptazinol valine carbamate oxymorphone-S-ile carbamate, oxymorphone-S-leu carbamate, oxymorphone-S-asp carbamate, oxymorphone-S-met carbamate, oxymorphone-S-his carbamate, oxymorphone-S-phe carbamate, oxymorphone-S-ser carbamate, hydromorphone-S-ile carbamate, hydromorphone-S-leu carbamate, hydromorphone-S-asp carbamate, hydromorphone-S-met carbamate, hydromorphone-S-his carbamate, hydromorphone-S-phe carbamate, hydromorphone-S-ser carbamate, buprenorphine-(S)-ile carbamate, buprenorphine-(S)-leu carbamate, buprenorphine-(S)-asp carbamate, buprenorphine-(S)-met carbamate, buprenorphine-(S)-his carbamate, buprenorphine-(S)-phe carbamate; buprenorphine-(S)-ser carbamate, meptazinol-(S)-ile carbamate, meptazinol-(S)-leu carbamate, meptazinol-(S)-asp carbamate, meptazinol-(S)-met carbamate, meptazinol-(S)-his carbamate, meptazinol-(S)-phe carbamate and meptazinol-(S)-ser carbamate, hydromorphone-S-val-val carbamate, hydromorphone-S-ile-ile, hydromorphone-S-leu-leu, oxymorphone-S-val-val carbamate, oxymorphone-S-ile-ile carbamate and oxymorphone-S-leu-leu carbamate, buprenorphine-(S)-val-val carbamate, buprenorphine-(S)-ile-ile carbamate, buprenorphine-(S)-leu-leu carbamate, meptazinol-(S)-val-val carbamate, meptazinol-(S)-ile-ile carbamate and meptazinol-(S)-leu-leu carbamate.
